SEQUENCE LISTING (4 sequences)

SEQ ID NO: 1 - DNA, 19 bp, Artificial sequence
  DNA Primer
agcatccacgtgttggctc

SEQ ID NO: 2 - DNA, 22 bp, Artificial sequence
  DNA primer
ccagcctactcattgggatcat

SEQ ID NO: 3 - DNA, 19 bp, Artificial sequence
  DNA primer
accctaaggccaaccgtga

SEQ ID NO: 4 - DNA, 21 bp, Artificial sequence
  DNA primer
cagaggcatacagggacagca

(19) United States
(12) Patent Application Publication
(10) Pub. No.: US 2021/0093565 A1
(43) Pub. Date: Apr. 1, 2021

(54) VESICLE-ENCAPSULATED SOCS

(71) Applicant: The Regents of the University of Michigan, Ann Arbor, MI (US)

(72) Inventors: Marc Peters-Golden, Ann Arbor, MI (US); James J. Moon, Ann Arbor, MI (US)

(21) Appl. No.: 16/071,290

(22) PCT Filed: Jan. 19, 2017
(86) PCT No.: PCT/US17/14070

§ 371 Date: Jul. 19, 2018

Related U.S. Application Data
(60) Provisional application No. 62/280,418, filed on Jan. 19, 2016.
(60) Provisional application No. 62/286,135, filed on Jan. 22, 2016.

(51) Int. Cl. A61K 9/127; A61K 9/00; A61K 38/17

(52) U.S. Cl. CPC A61K 9/127; A61K 38/1709; A61K 9/007

(57) ABSTRACT

Provided herein are synthetic vesicles carrying a payload of one or more suppressors of cytokine signaling (SOCS) proteins. In particular, liposomes encapsulating SOCS1 and/or SOCS3, and methods of delivery and use for the treatment of lung disease and conditions, are provided.

### CROSS REFERENCE TO RELATED APPLICATIONS

The present invention is a § 371 U.S. National Entry Application of PCT/US2017/014070, filed Jan. 19, 2017, which claims the priority benefit of U.S. Provisional Patent Application 62/280,418, filed Jan. 19, 2016 and U.S. Provisional Patent Application 62/286,135, filed Jan. 22, 2016, each of which are incorporated herein by reference in their entireties.

### STATEMENT REGARDING FEDERAL FUNDING

This invention was made with government support under grant number HL125555 awarded by the National Institutes of Health. The government has certain rights in the invention.

### SEQUENCE LISTING

The text of the computer readable sequence listing filed herewith, titled “34694-253_SEQUENCE_LISTING_ST25”, created Nov. 30, 2020, having a file size of 2,000 bytes, is hereby incorporated by reference in its entirety.

### FIELD

Provided herein are synthetic vesicles carrying a payload of one or more suppressors of cytokine signaling (SOCS) proteins. In particular, liposomes encapsulating SOCS1 and/or SOCS3, and methods of delivery and use for the treatment of lung disease and conditions, are provided.

### BACKGROUND

Although the lung is continuously exposed to foreign microbes, toxins, and allergens, it must limit inflammatory responses to these substances in order to preserve normal gas exchange. This requires the existence of natural endogenous brakes on inflammation, and implies that inflammatory diseases may be facilitated when these brakes are disrupted.

Many cytokines and growth factor receptors signal via the JAK-STAT pathway. The kinase JAK phosphorylates the transcription factor STAT, which then dimerizes and translocates into the nucleus to bind to target genes which carry out the inflammatory or mitogenic program. Suppressor of Cytokine Signaling (SOCS) proteins are endogenous brakes on JAK-STAT signaling; however, prior to the experimental evidence and embodiments presented herein, they had never been identified extracellularly.

### SUMMARY

Provided herein are synthetic vesicles carrying a payload of one or more suppressors of cytokine signaling (SOCS) proteins. In particular, liposomes encapsulating SOCS1 and/or SOCS3, and methods of delivery and use for the treatment of lung disease and conditions (e.g., diseases or conditions resulting from or causing inflammation, cancer, etc.), are provided.

In some embodiments, provided herein are compositions comprising synthetic vesicles encapsulating one or more SOCS polypeptides. In some embodiments, the synthetic vesicles are liposomes (e.g., MP-like, exo-like, etc.) encapsulating SOCS1, SOCS3, and/or active variants and/or fragments thereof. In some embodiments, in addition to SOCS1 and/or SOCS3 polypeptides, the vesicles encapsulate not more than 10 additional peptide or polypeptide species (e.g., 0, 1, 2, 3, 4, 5, 6, 7, 8, 9, 10).

In some embodiments, the synthetic vesicles (e.g., MP-like, exo-like, etc.) comprise one or more lipids selected from the group consisting of egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholipids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethyl ammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (PIs), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixtures thereof.

In some embodiments, the one or more SOCS polypeptides comprises a SOCS1 polypeptide. In some embodiments, the SOCS1 polypeptide comprises greater than 60% (e.g., 65%, 70%, 75%, 80%, 85%, 90%, 95%, 96%, 97%, 98%, 99%) sequence identity to SEQ ID NO: 1. In some embodiments, the SOCS1 polypeptide comprises less than 100% sequence identity to SEQ ID NO: 1.

In some embodiments, the one or more SOCS polypeptides comprises a SOCS3 polypeptide. In some embodiments, the SOCS3 polypeptide comprises greater than 60% (e.g., 65%, 70%, 75%, 80%, 85%, 90%, 95%, 96%, 97%, 98%, 99%) sequence identity to SEQ ID NO: 3. In some embodiments, the SOCS3 polypeptide comprises less than 100% sequence identity to SEQ ID NO: 3.

In some embodiments, the one or more SOCS polypeptides attenuate STAT phosphorylation.

In some embodiments, the synthetic vesicles are liposomes. In some embodiments, the synthetic vesicles are MP-like. In some embodiments, the synthetic vesicles are Exo-like.

In some embodiments, the synthetic vesicles are formulated for pulmonary administration. In some embodiments, the synthetic vesicles are formulated for inhalation by a subject. In some embodiments, the synthetic vesicles are aerosolized.

In some embodiments, provided herein are methods of treating or preventing a pulmonary condition or disease comprising administering a composition comprising vesicle-encapsulated SOCS polypeptides to a subject suffering from the pulmonary condition or disease. In some embodiments, the pulmonary condition or disease is characterized by inflammation. In some embodiments, administration of a composition comprising vesicle-encapsulated SOCS polypeptides results in decreased inflammation.

In some embodiments, provided herein are methods of treating lung cancer comprising administering a composition comprising vesicle-encapsulated SOCS polypeptides to a subject suffering from lung cancer.

### BRIEF DESCRIPTION OF THE DRAWINGS

FIG. 1. SOCS3 protein mediates inhibition of alveolar epithelial cell (AEC) STAT activation by alveolar macrophage (AM)-derived conditioned media (CM). (A, B) AECs were incubated for 2 h with medium alone (−) or CM obtained from AMs cultured overnight (+), challenged for 1 h with 20 ng/ml IL-6 (A) or 5 ng/ml IFNγ (B), and lysates analyzed for p-STAT3 (A) or p-STAT1 (B); activation is expressed as a percentage of the level of p-STAT3 (normalized to total STAT3) or p-STAT1 (normalized to β actin) measured in cytokine-treated cells not pretreated with CM. (C) SecretomeP 2.0-derived neural network scores for SOCS family members; those with scores >0.5 are predicted to be unconventionally secreted. (D) Overnight AM CM (+) or RPMI 1640 alone (−) were concentrated and subjected to WB analysis for SOCS3; bar graph depicts arbitrary densitometric units of SOCS3. (E) Cell lysates and CM from AMs incubated with non-targeting control (CTR) or SOCS3 siRNA were analyzed for SOCS3 protein by WB; representative blots are shown at top and mean lysate data are shown below. (F) AECs were incubated for 2 h with overnight CM obtained from untreated, CTR siRNA- or SOCS3 siRNA-treated AMs and then challenged with IL-6. STAT3 activation was assessed by determining phospho-STAT3 levels by WB; values in (F) represent the percentage of STAT3 activation present in unstimulated cells, which is indicated by the dashed line.

FIG. 2. SOCS3 secretion by AMs proceeds via an unconventional vesicular pathway and mainly involves MPs. (A) AMs were adhered and cultured for 1 h at 37° C. or at 4° C. Then CM was concentrated and subjected to WB analysis for SOCS3. SOCS3 levels in CM are expressed as the percent of SOCS3 secreted by AMs kept at 37° C. (B) AMs were treated with monensin (1 μM) for 1 h, after which CM was harvested for determination of TNF by ELISA (left), or concentrated and subjected to WB analysis for SOCS3 (right). SOCS3 levels in CM are expressed as arbitrary densitometric units. (C) CM was obtained from AMs after 1 h adherence, concentrated, and incubated for 2 h with 0.1 mg/mL proteinase K in the presence or absence of 1% Triton X-100, and then analyzed by WB for SOCS3. SOCS3 is expressed as the percentage of that measured in non-detergent-treated CM. The dashed vertical line separates lanes that were loaded on the same gel but were not contiguous. (D) Neat CM and the flow-through from a 0.2 μm filter were concentrated and subjected to either WB for SOCS3 or analysis by flow cytometry. Particles were further subjected to size determination using standard beads of known size. Additionally, MPs and Exos were purified from CM by differential centrifugation and subjected to WB for SOCS3. MPs were further analyzed for staining with FITC-annexin V and FITC-anti-SOCS3 with (continuous line) or without (dashed line) pretreatment with 0.2% NP-40. Additionally, whole CM, MPs, Exos and vesicle-free CM (VFCM) were collected and then subjected to SOCS3 quantitation by ELISA (bottom graph). (E) AM plasma membranes were labeled by incubating cells on ice in the dark for 20 min with 100 μM of the fluorescent lipid 18:1-06:0 NBD PC (green) and counterstained with DAPI; then cells were washed twice with PBS, plated for 1 h and MP budding assessed by fluorescence microscopy using a Nikon Eclipse E600 Microscope and 100× magnification arrows indicate membrane blebs. (F) The MP pellet from AM CM was incubated with FITC-annexin V in the dark and imaged on a Nikon TE300 with a 60× oil immersion objective (NA 1.40, total magnification of 600×).

FIG. 3. Uptake of SOCS3-containing MPs by AECs inhibits target cell STAT3 activation. (A) AECs were pretreated with CM from AMs cultured overnight for the time intervals indicated, after which they were challenged with IL-6 for 1 h and lysates subjected to WB for STAT3 activation; results are expressed as the percentage of the stimulated increase in cytokine-treated cells not receiving AM CM, indicated by the dashed line. (B) AECs were treated with or without AM CM for 2 h at 37° C. or at 4° C., after which AEC lysate proteins were subjected to SOCS3 quantitation by ELISA. Data are expressed as ng perm of total protein. (C) AM-derived MPs were labeled with FITC-annexin V and added to AECs at a ratio of 10:1 for 1 h at 37° C. or at 4° C. Increases in fluorescence in AEC cultures were determined by flow cytometry, and are depicted as histograms from a representative experiment (left) and mean fluorescence intensity (MFI; fold change versus background fluorescence of AECs alone) from 3 experiments (right). MFI of AECs receiving FITC-annexin V without MPs at 37° C. was similar to background (not shown). (D) MPs isolated from AM CM were incubated with AECs at a ratio of 10:1 for 2 h prior to stimulation with IL-6, lysates analyzed for STAT3 activation, expressed as the percentage of that determined in cytokine-treated AECs not pretreated with MPs. (E) AECs were pretreated with or without CM or with MP-depleted CM for 2 h at 37° C. prior to stimulation with IL-6, after which lysates were analyzed for STAT3 activation, expressed as the percentage of that determined in cytokine-treated AECs not pretreated with CM.

FIG. 4. SOCS1 protein is secreted in Exos and exerts inhibitory effects on AEC STAT1 activation. (A) Overnight AM CM (+) or RPMI 1640 alone (−) were concentrated and subjected to WB analysis for SOCS1; bar graph depicts arbitrary densitometric units of SOCS1. (B) MPs and Exos were isolated from overnight CM and subjected to WB for SOCS1. (C) AECs were pretreated for 2 h with (+) Exos isolated from overnight CM or with RPMI 1640 alone (−) prior to a 1-h stimulation with IFNγ, after which AEC lysate proteins were subjected to immunoblot analysis for p-STAT1. STAT1 activation was expressed as the percentage of p-STAT1, normalized for β actin, in cytokine-treated AECs not pretreated with AM-derived exosomes.

FIG. 5. SOCS3 secretion is a regulated phenomenon in vitro. (A) AMs were adhered to tissue culture plates for 60 min (adh) and then cultured for another 60 min after changing the medium (post-adh); SOCS3 in concentrated CM was analyzed by WB (top) and MP number was assessed by flow cytometry (bottom) and expressed as the percentage of the number quantified in 60-min post-adh CM. (B) AMs were adhered for the time intervals shown and SOCS3 in concentrated CM determined by WB. (C, D) Post-adh AMs were treated either with 1 μM PGE2 for the times indicated (C), or with 10 ng/ml IL-10 or 5 μg/ml LPS for 1 h (D), after which CM was concentrated and SOCS3 determined. SOCS3 levels are expressed as the percent of SOCS3 secreted following 60 min treatment with PGE2 (C) or as arbitrary densitometric units (D). The dashed vertical line in (C) separates lanes on the same gel that were not contiguous. (E) Post-adh AMs were treated for 1 h with PGE2, IL-10 or LPS at doses noted above; MP number in CM was assessed by flow cytometry (left) and the ratio of SOCS3 (determined by WB)/MP number is indicated (right).

FIG. 6. Expression and secretion of SOCS3 by various cell populations. (A) AMs obtained by BAL from normal human subjects were adhered and cultured for 1 h, and concentrated CM was analyzed by WB for SOCS3 (top) and SOCS1 (bottom); each lane represents an individual subject. (B) AMs and peritoneal macrophages (PMs) from a single mouse were cultured overnight and SOCS3 was determined by WB in concentrated CM and cell lysates. (C) AMs and PMs from a single rat were cultured overnight and SOCS3 was determined by WB in concentrated CM and cell lysates (left); data in graph are for lysate values and are expressed as a percentage of the level of SOCS3 (normalized to actin) measured in AMs; MPs were isolated from PM-derived CM and analyzed for SOCS3 staining following permeabilization with 0.2% NP-40 (right). (D) Bone marrow-derived macrophages obtained by in vitro differentiation of rat bone marrow cells for 6 days were re-adhered, their medium replaced, and CM obtained following culture for an additional 1 h; SOCS3 was analyzed following concentration of CM. (E) CCL-210 normal human lung fibroblasts were plated for 24 h, the medium changed, and subsequently cultured for an additional 24 h, after which cell lysates and concentrated CM were subjected to WB analysis for SOCS3; the dashed vertical line separates lanes from the same gel that were not contiguous. (F) Rat AEC lines L2 and RLE-6TN as well as rat AMs were cultured for 16 h. Lysates were analyzed by WB for SOCS3. The dashed vertical line separates lanes that were on the same gel but were not contiguous.

FIG. 7. AM-derived SOCS attenuates pulmonary STAT activation in vivo. (A-D) Mouse lungs were pretreated oropharyngeally with 50 μl of saline alone or saline containing ˜3×10⁶ MPs isolated from CM from AMs (A-D) or peritoneal macrophages (PMs) (A, C). 2 h later, mice received an oropharyngeal dose of 50 μl of saline alone or saline containing 0.1 μg IFNγ. 1 h thereafter their AMs were removed by lavage, and lung homogenates were prepared from the middle right lung for analysis of p-STAT1 (A) and p-STAT3 (B) by WB, and from the inferior right lung for analysis of MCP-1 mRNA by q-RT-PCR (C). p-STAT1 levels in lysates of lavaged AMs were analyzed by WB (D). (E) Mice were treated with intrapulmonary saline alone or saline containing AM MPs prior to IFNγ, as in (A), and lung sections prepared from the left lung were incubated with hematoxylin to stain nuclei blue and anti-pSTAT1 followed by DAB to stain p-STAT1 red; photographs were taken using a Nikon Eclipse E600 Microscope (40× magnification), and insets represent enlarged images (top); p-STAT1 staining was quantified by first separating the colors using color deconvolution plugin (Image J software) and performing densitometric analysis of red staining (bottom) in 10 randomly-selected fields, which was expressed relative to the area of the whole field. Scale bars equal 500 μm.

FIG. 8. SOCS secretion in the lung in vivo is regulated by immunomodulatory substances and dysregulated in association with cigarette smoking. (A) Mice (3 mice per group) were subjected to intrapulmonary administration of 50 μl of saline alone or saline containing 15 μg PGE2 and/or LPS. BALF was harvested 3 h later, pooled, concentrated, and subjected to WB analysis for SOCS3. (B) BALF from never smokers or healthy current smokers was concentrated and subjected to WB analysis for SOCS3 and SOCS1; results from 3 subjects per group are depicted, with each lane representing an individual subject (top); following densitometric analysis of blots, SOCS levels in BALF of smokers was expressed as a percentage of that in never smokers (bottom, left). SOCS3 levels were also determined by ELISA of sonicated BALF (bottom, right); the mean level in never smokers was 0.26±0.12 pg/μg protein, and that in smokers was expressed as a percentage of the never smoker level. (C) Mice were subjected or not to 2 h/d of cigarette smoke for 3 or 7 d, and BALF was subjected to WB analysis for SOCS3; data at bottom represent mean±SE arbitrary densitometric units. *P<0.05 vs. human never smokers (B) or unexposed mice (C).

FIG. 9. SOCS3 secretion is decreased in KRAS lung cancer BALF and AM-CM. (A) SOCS3 levels in BALF measured by ELISA. (B) SOCS3 levels in BALF measured by western blot. (C) Total MP numbers in AM-CM per 500,000 cells. (D) SOCS3 levels in AM-CM after 24 hour culture. E) SOCS3 protein in AM lysates measured by western blot (n=4). F) PGE2 levels in BALF measured by ELISA.

FIG. 10. Human A549 adenocarcinoma cells or rat L2 alveolar epithelial cells were cultured with PKH dye-labelled MPs for 2 hours. The cells were trypsinized and analyzed for fluorescence via flow cytometry. Uptake of PKH MPs was determined by mean-fluorescence intensity shift compared to untreated control cells.

FIG. 11. Human A549 adenocarcinoma cells were cultured with rat AM-CM for 2 hours. They were then stimulated with 10 ng/mL recombinant human IL-6 for 1 hour and lysates were collected and analyzed for phospho-STAT3 (Tyr705) by western blot.

FIG. 12. (Top) Liposome loading with recombinant SOCS3. Serial dilutions of 400 nm and 50 nm liposomes loaded with SOCS3 were analyzed by Western blot. (Bottom) Effect of empty or SOCS3-containing liposomes on IL-6-induced STAT3 phosphorylation in normal alveolar L2 cells.

FIG. 13. Effect of liposomes on IL-6 induced STAT3 phosphorylation in A549 adenocarcinoma cells. Data are expressed as percent inhibition of STAT3 phosphorylation elicited by 50 nm or 400 nm SOCS3-containing liposomes.

FIG. 14. Effect of liposomal SOCS3 on transcription factor activation in bronchial epithelial BEAS-2b cells. Cells were pretreated with empty or SOCS3-containing liposomes of 400 nm diameter, then stimulated with IL-4/IL-13. Activation (phosphorylation) of STATE (left) and NF-kB (right) were determined by Western blotting and normalized for total actin. Values are expressed relative to those in the empty liposome condition, taken as 100%.

FIG. 15. Effect of liposomal SOCS3 on STAT3 activation in bronchial epithelial BEAS-2b cells. Cells were pretreated with empty or SOCS3-containing liposomes of 400 nm (left) or 50 nm diameter (right), then stimulated with IL-4/IL-13. Activation (phosphorylation) of STAT3 was determined by Western blotting and normalized for total STAT3. Values are expressed relative to those in the empty liposome condition, taken as 100%.

FIG. 16. Effect of liposomal SOCS3 on IL-13/TNFα-induced eotaxin-1 protein in BEAS-2b cells. Cells were pretreated for 2 hours with 50 nm empty or SOCS3-containing liposomes, then stimulated with cytokines and conditioned medium analyzed 2 hours later for eotaxin-1 protein by ELISA.

FIG. 17. Effect of liposomal SOCS3 on cell proliferation in A549 adenocarcinoma cells. Cells were treated with medium alone (control), 50 nm empty (E50) or SOCS3-containing liposomes (S50), or microvesicles from primary alveolar macrophages (AM MV). Cell proliferation was assessed by CyQuant DNA dye-binding assay 72 hours later. Data from each graph represents a separate experiment, with each value representing the mean and SE from triplicate determinations. The dashed line indicates the control level of proliferation.

FIG. 18. Effect of SOCS3 liposomes on A549 adenocarcinoma cell apoptosis. Cells were treated with medium alone (control), the apoptosis inducer Fas ligand (FasL), empty 50 nm liposomes, of SOCS3-containing 50 nm liposomes. After 24 and 48 hours, cells were harvested and analyzed by flow cytometry for annexin-V surface staining. The percent of cells staining positively (i.e., apoptotic cells) is displayed.

FIG. 19. In vivo effect of SOCS3-containing liposomes on cytokine-induced inflammatory responses in the mouse lung. 50 nm empty or SOCS3 liposomes were administered to the lung of C57BL/6 mice; 2 hours later IFN-γ was administered, and 1 hour later, lungs were harvested and homogenates analyzed by Western blot for phospho-STAT1 (left) and by RT-PCR for mRNA levels of the STAT1-dependent chemokine gene, IP10 (right). Values represent mean and SE from 2 mice. Dashed line represents the value observed with empty liposomes.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. Thus, for example, reference to “a SOCS polypeptide” is a reference to one or more SOCS polypeptides and equivalents thereof known to those skilled in the art, and so forth.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the term “vesicle” refers to any small enclosed structures. Often the structures are membranes composed of lipids, proteins, glycolipids, steroids or other components associated with membranes. Vesicles can be naturally generated (e.g., the vesicles present in the cytoplasm of cells that transport molecules and partition specific cellular functions) or can be synthetic (e.g., liposomes).

As used herein, the term “exosome” (“Exo”) refers to a subset of vesicles that are formed intracellularly and are released from cells following the exocytic fusion of intracellular multivesicular bodies with the plasma membrane. Exosomes are typically on the order of 20-100 nm in diameter (e.g., 20 nm, 30 nm, 40 nm, 50 nm, 60 nm, 70 nm, 80 nm, 90 nm, 100 nm, or suitable ranges there between).

As used herein, the terms “microparticle” (“MP”), “ectosome”, and “microvesicle” synonymously refer to a subset of vesicles having typical diameters between about 80 and 1200 nm (e.g., 80 nm, 90 nm, 100 nm, 120 nm, 140 nm, 160 nm, 180 nm, 200 nm, 250 nm, 300 nm, 350 nm, 400 nm, 450 nm, 500 nm, 600 nm, 700 nm, 800 nm, 900 nm, 1000 nm, 1100 nm, 1200 nm, or suitable ranges there between). Microparticles are released from cells by budding or shedding.

As used herein, the term “liposome” refers to artificially-produced lipid complexes (e.g., spherical in shape) that are induced to segregate out of aqueous media. Liposomes are synthetic vesicles composed of amphiphilic lipids arranged in a spherical bilayer or bilayers. Liposomes may be “MP-like”, having characteristics (e.g., size, membrane composition, etc.) similar to microparticles; “Exo-like”, having characteristics (e.g., size, membrane composition, etc.) similar to exosomes; or of any suitable size and composition. Liposomes are unilamellar or multilamellar vesicles which have a membrane formed from a lipophilic material and an aqueous interior that contains the composition to be delivered. Liposomes range in diameter from 20 nm to about 3 μm (e.g., 20 nm, 30 nm, 40 nm, 50 nm, 60 nm, 70 nm, 80 nm, 90 nm, 100 nm, 120 nm, 140 nm, 160 nm, 180 nm, 200 nm, 250 nm, 300 nm, 350 nm, 400 nm, 450 nm, 500 nm, 600 nm, 700 nm, 800 nm, 900 nm, 1000 nm, 1500 nm, 2000 nm, 2500 nm, 3000 nm, or suitable ranges there between). “Small unilaminar liposomes” are typically on the order of 20-100 nm in diameter, “large unilaminar liposomes” are typically on the order of 100-500 nm in diameter, “giant unilaminar liposomes” are typically on the order of 500 nm in diameter and larger, and “large multilaminar liposomes are typically on the order of 200-3000 nm in diameter. Liposomes are synthetically prepared from a defined amphipathic lipid or set of amphipathic lipids (e.g., phospholipids) and a defined polar solvent (e.g., aqueous solvent, water).

As used herein, the term “synthetic” refers to compositions and systems that are designed or prepared by man. For example, a synthetic protein or nucleic acid is one that is produced by man or the production of which is induced by man. A synthetic vesicle is one prepared by man, having a bilayer of defined composition and defined contents.

As used herein, the term “artificial” refers to compositions and systems that are not naturally occurring. For example, an artificial polypeptide (e.g., SOCS1 or SOCS3) or nucleic acid is one comprising a non-natural sequence (e.g., a polypeptide without 100% identity with a naturally-occurring protein or a fragment thereof). An artificial liposome is one having a bilayer composition and/or payload that is not naturally occurring. For example, in some embodiments, an artificial liposome comprises only a small number of different cargos (e.g., single-cargo liposomes (e.g., SOCS1 or SOCS3), two-cargo liposomes, (e.g., SOCS1 and SOCS3), 3 cargo species, 4 cargo species, 5 cargo species, 6 cargo species, 7 cargo species, 8 cargo species, 9 cargo species, 10 cargo species, 20 cargo species, 30 cargo species, 40 cargo species, 50 cargo species, or ranges there between). The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “mutant polypeptide” refers to a variant of a polypeptide having a distinct amino acid sequence from the most common variant occurring in nature, referred to as the “wild-type” sequence. A mutant polypeptide may be a naturally-occurring protein that is not the most common sequence in nature (or a subsequence thereof), or may be a polypeptide that is not a naturally-occurring sequence (or a subsequence thereof). For example, a “mutant SOCS3” may be a naturally-occurring, non-wild-type SOCS3, or may be a synthetic SOCS3 that does not occur in nature.

As used herein, the term “synthetic polypeptide”, consistent with the definition of “synthetic” above, refers to a polypeptide that is produced by human manipulation (e.g., human design and/or human involvement in preparation).

As used herein, the term “artificial polypeptide”, consistent with the definition of “artificial” above, refers to a polypeptide that has a distinct amino acid sequence from those found in natural peptides and/or proteins. An artificial protein is not a subsequence of a naturally occurring protein, either the wild-type (i.e., most abundant) or mutant versions thereof. For example, a “artificial SOCS1” (“aSOCS1”) is not a subsequence of a naturally occurring SOCS1 sequence. An “artificial polypeptide,” as used herein, may be produced or synthesized by any suitable method (e.g., recombinant expression, chemical synthesis, enzymatic synthesis, etc.).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:

1) Alanine (A) and Glycine (G);

2) Aspartic acid (D) and Glutamic acid (E);

3) Asparagine (N) and Glutamine (Q);

4) Arginine (R) and Lysine (K);

5) Isoleucine (I), Leucine (L), Methionine (M), and Valine (V);

6) Phenylalanine (F), Tyrosine (Y), and Tryptophan (W);

7) Serine (S) and Threonine (T); and

8) Cysteine (C) and Methionine (M).

Naturally occurring residues may be divided into classes based on common side chain properties, for example: polar positive (histidine (H), lysine (K), and arginine (R)); polar negative (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics (e.g., chemically modified peptides, peptoids (side chains are appended to the nitrogen atom of the peptide backbone, rather than to the α-carbons), β-peptides (amino group bonded to the β carbon rather than the α carbon), etc.) and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

As used herein, the term “sequence identity” refers to the degree to which two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) differ only by conservative and/or semi-conservative amino acid substitutions. The “percent sequence identity” (or “percent sequence similarity”) is calculated by: (1) comparing two optimally aligned sequences over a window of comparison (e.g., the length of the longer sequence, the length of the shorter sequence, a specified window, etc.), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “subject” broadly refers to any animal, including but not limited to, human and non-human animals (e.g., dogs, cats, cows, horses, sheep, poultry, fish, crustaceans, etc.). As used herein, the term “patient” typically refers to a subject that is being treated for a disease or condition.

As used herein, the term “effective amount” refers to the amount of a composition (e.g., vesicle-encapsulated SOCS) sufficient to effect beneficial or desired results. An effective amount can be administered in one or more administrations, applications or dosages and is not intended to be limited to a particular formulation or administration route.

As used herein, the terms “administration” and “administering” refer to the act of giving a drug, prodrug, or other agent, or therapeutic treatment (e.g., artificial peptide) to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intravenously, subcutaneously, intratumorally, intraperitoneally, etc.) and the like. Routes of particular import to embodiments herein include oral and/or nasal inhalation (e.g., of an aerosolized therapeutic).

As used herein, the terms “co-administration” and “co-administering” refer to the administration of at least two agents or compositions (e.g., vesicle-encapsulated SOCS and one or more additional therapeutics or therapies) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “treatment” means an approach to obtaining a beneficial or intended clinical result. The beneficial or intended clinical result may include alleviation of symptoms, a reduction in the severity of the disease, inhibiting an underlying cause of a disease or condition, stabilizing diseases in a non-advanced state, preventing disease progression, delaying the progress of a disease, and/or improvement or alleviation of disease conditions.

As used herein, the term “pharmaceutical composition” refers to the combination of an active agent (e.g., vesicle-encapsulated SOCS) with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety. Carriers that find use with liposome delivery and/or preparation or delivery of aerosols are of particular use herein.

Many embodiments herein are described using open “comprising” language. Such embodiments encompass multiple closed “consisting of” and/or “consisting essentially of” embodiments, which may alternatively be claimed or described using such language.

### DETAILED DESCRIPTION

Provided herein are synthetic vesicles carrying a payload of one or more suppressors of cytokine signaling (SOCS) polypeptides. In particular, liposomes encapsulating SOCS1 and/or SOCS3, and methods of delivery and use for the treatment of lung disease and conditions, are provided.

Experiments conducted during development of embodiments herein demonstrated that AMs from humans and rodents constitutively secrete SOCS1 and SOCS3 proteins and that relevant bioactive molecules tune secretion up or down within minutes. SOCS1 and 3 are secreted within specific types of vesicles, namely Exos and MPs, respectively, which are taken up by AECs to inhibit cytokine-induced STAT activation in vitro and in vivo.

Experiments conducted during development of embodiments herein also demonstrated SOCS3 levels detected in lung lining fluid are diminished in the mutant KRAS mouse model of lung cancer; this is despite markedly elevated levels of prostaglandin E2 (PGE2) in the KRAS mice. In normal lungs and in cultured AMs it has been observed that PGE2 potentiates secretion of SOCS3. AMs isolated by lavage from these tumor-bearing lungs and placed into culture demonstrate no difference from those of non-tumor-bearing control lungs in intracellular levels of SOCS3, but exhibit a dramatic reduction in their basal ability to secrete SOCS3. Furthermore, the ability of A549 lung cancer cells to take up fluorescently labeled AM-derived microparticles has been tested, and microparticle uptake by cancer cells is not only preserved relative to normal epithelial cells, but is actually enhanced. Incubation of A549 cells with SOCS3-AM-derived conditioned medium (which contains microparticles) attenuates the increase in phosphorylated (activated) STAT3 in response to treatment with the cytokine IL-6.

In some embodiments, artificial vesicles (liposomes) encapsulating recombinant SOCS proteins are administered for therapeutic purposes. In some embodiments, such vesicles are created by mixing artificial SOCS1 and/or SOCS3 polypeptides with phospholipids (e.g., pure phospholipids). In some embodiments, since natural vesicles contain numerous other forms of molecular cargo in addition to SOCS, the artificial vesicles encapsulating SOCS as their only cargo produce more predictable effects. Therapeutic application of such SOCS1 and/or SOCS3 vesicles finds use in, for example inflammatory lung diseases (e.g., pneumonitis, asthma, COPD) and lung cancer, due to the pivotal role played by STAT activation in both of these scenarios. In both of these contexts, small molecule JAK inhibitors are either FDA-approved or in development. The potential value of SOCS administration in order to inhibit STAT activation is underscored by the recognition that: (1) SOCS expression is quite low in normal respiratory epithelial cells; (2) its secretion by alveolar macrophages is inhibited by cigarette smoke and other pro-inflammatory substances; and (3) SOCS is frequently either mutated or epigenetically silenced in many cancers. In addition to avoidance of potential drug side effects, another advantage of liposomal SOCS protein administration as a therapy over JAK inhibitors is that SOCS proteins inhibit signaling pathways other than JAK-STAT, such as MAP kinases and NF-kB, thereby providing broader anti-inflammatory actions.

In some embodiments, provided herein are compositions comprising vesicle-encapsulated SOCS polypeptides (e.g., SOCS1 and/or SOCS3 polypeptides), and methods of preparation and use thereof for treating lung disease and conditions. In some embodiments, SOCS1 polypeptides are encapsulated within an Exo-like vesicle or a non-Exo-like vesicle. In some embodiments, SOCS3 polypeptides are encapsulated within an MP-like vesicle or a non-MP-like vesicle. In some embodiments, compositions comprise naturally-occurring and/or artificial SOCS1 and/or SOCS3 polypeptides (e.g., synthetically produced). A SOCS1 or SOCS3 polypeptide may comprise a fragment or mutant polypeptide retaining all or a portion of the activity (e.g., attenuating STAT phosphorylation) and/or structural characteristics of the natural SOCS1 or SOCS3. In some embodiments, compositions are formulated for administration to the lungs and/or respiratory system of a subject (e.g., human or non-human subject). In some embodiments, compositions are formulated for inhalation (e.g., aerosolized).

In some embodiments, provided herein are vesicle-encapsulated payloads for delivery to a cell, tissue, organ, system, subject, or other target. In some embodiments, the payload comprises a SOCS protein, polypeptide or peptide, or a variant and/or fragment thereof. In some embodiments, a SOCS polypeptide is a naturally occurring SOCS polypeptide (e.g., SOCS1 (SEQ ID NO:1), SOCS3 (SEQ ID NO:3), etc.) or a fragment thereof. In some embodiments, a SOCS polypeptide is a synthetic and/or artificial SOCS polypeptide capable of attenuating STAT phosphorylation (e.g., when administered to cells or tissues via a vesicle delivery system).

In some embodiments, compositions are provided comprising a SOCS1 polypeptide (e.g., encapsulated within a vesicle). The SOCS1 polypeptide may be a synthetic (e.g., designed my man) or naturally-occurring sequence. The SOCS1 polypeptide may be full-length (e.g., having significant sequence identity and/or similarity with a full-length naturally-occurring SOCS1) or a SOCS1 fragment. In embodiments in which a SOCS1 fragment or synthetic SOCS1 sequence (e.g., designed my man) is provided, the SOCS1 polypeptide retains one or more structural and/or functional features of the wild-type (or other naturally-occurring SOCS1), such as the capability to attenuate STAT phosphorylation, inhibit cytokine induction of STAT, suppress inflammatory gene expression, etc.

In some embodiments, a SOCS1 polypeptide comprises at least 60% sequence identity (e.g., 60%, 65%, 70%, 75%, 80%, 85%, 90%, 95%, 96%, 97%, 98%, 99%, 100%, or ranges there between) with all or a portion of wild-type SOCS1 (SEQ ID NO:1). In some embodiments, a SOCS1 polypeptide comprises less than 100% sequence identity with all or a portion of wild-type SOCS1 (SEQ ID NO:1). In some embodiments, a SOCS1 polypeptide comprises at least 60% sequence similarity (e.g., 60%, 65%, 70%, 75%, 80%, 85%, 90%, 95%, 96%, 97%, 98%, 99%, 100%, or ranges there between) with all or a portion of wild-type SOCS1 (SEQ ID NO:1). In some embodiments, a SOCS1 polypeptide comprises less than 100% sequence similarity with all or a portion of wild-type SOCS1 (SEQ ID NO:1). In some embodiments, a SOCS1 polypeptide is not a naturally-occurring SOCS1 variant (e.g., not the wild-type SOCS1 or a naturally-occurring variant thereof).

In some embodiments, a SOCS1 polypeptide is not a full-length SOCS1 protein (e.g., synthetic (e.g., designed my man) or naturally occurring. In some embodiments, a SOCS1 polypeptide is a fragment of a full-length SOCS1 (e.g., a fragment of SEQ ID NO:1), but maintains all or a portion of SOCS1 activity (e.g., the capability to attenuate STAT phosphorylation, inhibit cytokine induction of STAT, suppress inflammatory gene expression, etc.). In some embodiments, a SOCS1 polypeptide is an active SOCS1 peptide of 10-50 amino acids. In some embodiments, a SOCS1 polypeptide is 50-210 amino acids in length (e.g., 50, 60, 70, 80, 90, 100, 110, 120, 130, 140, 150, 160, 170, 180, 190, 200, 210, or ranges there between). In some embodiments, in addition to a SOCS1 sequence, a SOCS1 polypeptide is fused to one or more additional peptide or polypeptide sequences to impart stability, solubility, an additional activity, etc.

In some embodiments, a SOCS1 polypeptide comprises at least 1 mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60, or more, and any ranges therein) relative to a naturally-occurring SOCS1 (e.g. SEQ ID NO:1, all naturally-occurring SOCS1 proteins) over the length of the SOCS1 polypeptide. In some embodiments, a SOCS1 polypeptide comprises at least 1 non-conservative mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60, or more, and any ranges therein) from the wild-type or a natural SOCS1 sequence over the length of the polypeptide. In some embodiments, a SOCS1 polypeptide comprises at least 1 conservative mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60, or more, and any ranges therein) from the wild-type or a natural SOCS1 sequence over the length of the peptide. In some embodiments, a SOCS1 polypeptide comprises at least 1 semi-conservative mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60, or more, and any ranges therein) from the wild-type or a natural SOCS1 sequence over the length of the peptide. In some embodiments, a peptide or polypeptide is provided that comprises a synthetic SOCS1 polypeptide sequence. In some embodiments, a synthetic SOCS1 comprises a combination of conservative, semi-conservative and/or non-conservative substitutions relative to a wild-type and/or naturally-occurring SOCS1 sequence. In some embodiments, a synthetic SOCS1 comprises deletion of one or more segments relative to a wild-type and/or naturally-occurring SOCS1 sequence. In some embodiments, a synthetic SOCS1 comprises addition or insertion of one or more segments relative to a wild-type and/or naturally-occurring SOCS1 sequence.

In some embodiments, a SOCS1 polypeptide exhibits SOCS1 activity (e.g., the capability to attenuate STAT phosphorylation, inhibit cytokine induction of STAT, suppress inflammatory gene expression, etc.), for example, in one or more of the assays set forth in the Examples herein. In some embodiments, a SOCS1 polypeptide exhibits at least 50% of the activity of wild-type SOCS1 (SEQ ID NO:1). In some embodiments, a SOCS1 polypeptide exhibits enhanced activity (e.g., in one or more of the assays set forth in the Examples herein) relative to wild-type SOCS1 (SEQ ID NO:1). In some embodiments, the activity (e.g., the capability to attenuate STAT phosphorylation, inhibit cytokine induction of STAT, suppress inflammatory gene expression, etc.) of a SOCS1 polypeptide is increased by >10%, >20%, >30%, >40%, >50%, >60%, >70%, >80%, >90%, >2-fold, >3-fold, >4-fold, >5-fold, >6-fold, >8 fold, >10-fold, or >20-fold relative to SEQ ID NO: 1.

In some embodiments, compositions are provided comprising a SOCS3 polypeptide (e.g., encapsulated within a vesicle). The SOCS3 polypeptide may be a synthetic (e.g., designed my man) or naturally-occurring sequence. The SOCS3 polypeptide may be full-length (e.g., having significant sequence identity and/or similarity with a full-length naturally-occurring SOCS3) or a SOCS3 fragment. In embodiments in which a SOCS3 fragment or synthetic SOCS3 sequence is provided, the SOCS3 polypeptide retains one or more structural and/or functional features of the wild-type (or other naturally-occurring SOCS3), such as the capability to attenuate STAT phosphorylation, inhibit cytokine induction of STAT, suppress inflammatory gene expression, etc.

In some embodiments, a SOCS3 polypeptide comprises at least 60% sequence identity (e.g., 60%, 65%, 70%, 75%, 80%, 85%, 90%, 95%, 96%, 97%, 98%, 99%, 100%, or ranges there between) with all or a portion of wild-type SOCS3 (SEQ ID NO:3). In some embodiments, a SOCS3 polypeptide comprises less than 100% sequence identity with all or a portion of wild-type SOCS3 (SEQ ID NO:3). In some embodiments, a SOCS3 polypeptide comprises at least 60% sequence similarity (e.g., 60%, 65%, 70%, 75%, 80%, 85%, 90%, 95%, 96%, 97%, 98%, 99%, 100%, or ranges there between) with all or a portion of wild-type SOCS3 (SEQ ID NO:3). In some embodiments, a SOCS3 polypeptide comprises less than 100% sequence similarity with all or a portion of wild-type SOCS3 (SEQ ID NO:3). In some embodiments, a SOCS3 polypeptide is not a naturally-occurring SOCS3 variant (e.g., not the wild-type SOCS3 or a naturally-occurring variant thereof).

In some embodiments, a SOCS3 polypeptide is not a full-length SOCS3 protein (e.g., synthetic (e.g., designed my man) or naturally occurring. In some embodiments, a SOCS3 polypeptide is a fragment of a full-length SOCS3 (e.g., a fragment of SEQ ID NO:3), but maintains all or a portion of SOCS3 activity (e.g., the capability to attenuate STAT phosphorylation, inhibit cytokine induction of STAT, suppress inflammatory gene expression, etc.). In some embodiments, a SOCS3 polypeptide is an active SOCS3 peptide of 10-50 amino acids. In some embodiments, a SOCS3 polypeptide is 50-220 amino acids in length (e.g., 50, 60, 70, 80, 90, 100, 110, 120, 130, 140, 150, 160, 170, 180, 190, 200, 210, 215, 220, or ranges there between). In some embodiments, in addition to a SOCS3 sequence, a SOCS3 polypeptide is fused to one or more additional peptide or polypeptide sequences to impart stability, solubility, an additional activity, etc.

In some embodiments, a SOCS3 polypeptide comprises at least 1 mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60, or more, and any ranges therein) relative to a naturally-occurring SOCS3 (e.g. SEQ ID NO:3, all naturally-occurring SOCS3 proteins) over the length of the SOCS3 polypeptide. In some embodiments, a SOCS3 polypeptide comprises at least 1 non-conservative mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60, or more, and any ranges therein) from the wild-type or a natural SOCS3 sequence over the length of the polypeptide. In some embodiments, a SOCS3 polypeptide comprises at least 1 conservative mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60, or more, and any ranges therein) from the wild-type or a natural SOCS3 sequence over the length of the peptide. In some embodiments, a SOCS3 polypeptide comprises at least 1 semi-conservative mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 47, 48, 49, 50, 51, 52, 53, 54, 55, 56, 57, 58, 59, 60, or more, and any ranges therein) from the wild-type or a natural SOCS3 sequence over the length of the peptide. In some embodiments, a peptide or polypeptide is provided that comprises a synthetic a SOCS3 polypeptide sequence. In some embodiments, a synthetic SOCS3 comprises a combination of conservative, semi-conservative and/or non-conservative substitutions relative to a wild-type and/or naturally-occurring SOCS3 sequence. In some embodiments, a synthetic SOCS3 comprises deletion of one or more segments relative to a wild-type and/or naturally-occurring SOCS3 sequence. In some embodiments, a synthetic SOCS3 comprises addition or insertion of one or more segments relative to a wild-type and/or naturally-occurring SOCS3 sequence.

In some embodiments, a SOCS3 polypeptide exhibits SOCS3 activity (e.g., the capability to attenuate STAT phosphorylation, inhibit cytokine induction of STAT, suppress inflammatory gene expression, etc.), for example, in one or more of the assays set forth in the Examples herein. In some embodiments, a SOCS3 polypeptide exhibits at least 50% of the activity of wild-type SOCS3 (SEQ ID NO:3). In some embodiments, a SOCS3 polypeptide exhibits enhanced activity (e.g., in one or more of the assays set forth in the Examples herein) relative to wild-type SOCS3 (SEQ ID NO:3). In some embodiments, the activity (e.g., the capability to attenuate STAT phosphorylation, inhibit cytokine induction of STAT, suppress inflammatory gene expression, etc.) of a SOCS3 polypeptide is increased by >10%, >20%, >30%, >40%, >50%, >60%, >70%, >80%, >90%, >2-fold, >3-fold, >4-fold, >5-fold, >6-fold, >8 fold, >10-fold, or >20-fold relative to SEQ ID NO: 3.

In some embodiments, provided herein are compositions, formulations, and pharmaceutical preparations comprising vesicle-encapsulated SOCS polypeptides (e.g., liposome-encapsulated SOCS polypeptides). In some embodiments, the lipid portion of the vesicles/liposomes in embodiments herein are composed primarily of vesicle-forming lipids. Such a vesicle-forming lipid is one which (a) can form spontaneously into bilayer vesicles in water, or (b) is stably incorporated into lipid bilayers.

The vesicle-forming lipids finding use herein are typically ones having two hydrocarbon chains (e.g., acyl chains) and a head group, either polar or nonpolar. There are a variety of synthetic vesicle-forming lipids and naturally-occurring vesicle-forming lipids, including the phospholipids, such as phosphatidylcholine, phosphatidylethanolamine, phosphatidic acid, phosphatidylinositol, and sphingomyelin, where the two hydrocarbon chains are typically between about 14-22 carbon atoms in length, and have varying degrees of unsaturation. Lipids and phospholipids having acyl chains with varying degrees of saturation can be obtained commercially or prepared according to published methods. Other suitable lipids include glycolipids and sterols such as cholesterol.

In some embodiments, diacyl-chain lipids for use herein include diacyl glycerol, phosphatidyl ethanolamine (PE), diacylaminopropanediols, such as disteroylaminopropanediol (DS), and phosphatidylglycerol (PG). These lipids may find use in the liposome outer layer at a mole ratio between about 1-20 mole percent.

In some embodiments, a vesicle-forming lipid is selected to achieve a specified degree of fluidity or rigidity, to control the stability of the liposome and to control the rate of release of the liposome payload. The rigidity of a liposome may also play a role in fusion of the liposome to a target cell. Liposomes having a more rigid lipid bilayer, or a liquid crystalline bilayer, are achieved by incorporation of a relatively rigid lipid, such as a lipid having a relatively high phase transition temperature (e.g., >30° C., >35° C., >40° C., >45° C., >50° C., >55° C., >60° C.). Rigid (e.g. saturated) lipids contribute to greater membrane rigidity in the lipid bilayer. Other lipid components, such as cholesterol, are also known to contribute to membrane rigidity in lipid bilayer structures. Conversely, lipid fluidity is achieved by incorporation of a relatively fluid lipid, typically one having a lipid phase with a relatively low liquid to liquid-crystalline phase transition temperature (<30° C., <25° C., <20° C., <15° C.).

In some embodiments, all or a portion of the vesicle-forming lipids in a vesicle/liposome herein comprise phospholipids selected from dilauroylphatidylcholine (DLPC), dimyristoylphatidylcholine (DMPC), dipalmitoylphosphatidylcholine (DPPC), distearylphosphatidylcholine (DSPC), dioleylphosphatidylcholine (DOPC), dimyristoylphosphatidylethanolamine (DMPE), dipalmitoylphatidylethanolamine (DPPE), dioleylphosphatidylethanolamine (DOPE), dimyristoylphosphate (DMPA), dipalmitoylphosphate (DPPA), dioleylphosphate (DOPA), dimyristoylphosphoglycerol (DMPG), dipalmitoylphosphoglycerol (DPPG), dioleylphosphoglycerol (DOPG), dimyristoylphosphatidylserine (DMPS), dipalmitoylphosphatidylserine (DPPS), dioleylphosphatidylserine (DOPS), etc.

In some embodiments, a portion of the vesicle-forming lipids are sphingoglycolipid, glyceroglycolipid, or other suitable lipids.

In some embodiments, a vesicle formulation comprises a lipid selected from the group consisting of egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (REPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholipids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethyl ammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (PIs), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixtures thereof.

In some embodiments, liposome are prepared in accordance with field-accepted methods (e.g., sonication, extrusion, the Mozafari method (Micron (Oxford, England: 1993) 38 (8): 841-7.; herein incorporated by reference in its entirety), etc.). For example, the methods described in Liposome Technology, vol. 1, 2nd edition (by Gregory Gregoriadis (CRC Press, Boca Raton, Ann Arbor, London, Tokyo), Chapter 4, pp 67-80, Chapter 10, pp 167-184 and Chapter 17, pp 261-276 (1993)), which is herein incorporated by reference in its entirety, are used. Suitable methods employ techniques including, but not limited to, sonication, ethanol injection, French press, ether injection, cholic acid treatment, a calcium fusion, a lyophilization, reverse phase evaporation, and combinations thereof.

The size of the vesicles/liposomes herein is not particularly limited, unless specifically stated, and is typically between 30 and 600 nm (e.g., 30 nm, 40 nm, 50 nm, 60 nm, 70 nm, 80 nm, 90 nm, 100 nm, 120 nm, 140 nm, 160 nm, 180 nm, 200 nm, 250 nm, 300 nm, 350 nm, 400 nm, 450 nm, 500 nm, 550 nm, 600 nm, and ranges there between (e.g., 30-70 nm, 200-600 nm, etc.)). The structure of the liposomes is not particularly limited, unless specifically stated, and may be unilamellar, multilamellar, or combinations thereof.

The solution encapsulated with the liposomes typically comprises an aqueous solution, such as, but not limited to physiologically suitable buffers, saline, water, a water soluble organic solvent (e.g., glycerine), and combinations thereof.

In some embodiments, inclusion of a payload into the liposome interior can be performed by ordinary methods.

In some embodiments, provided herein are methods of treating inflammation or inflammation-associated diseases or conditions (e.g., in the lung, etc.) by delivery (e.g., via aerosol or gavage) of a liposomal composition comprising a natural or synthetic SOCS polypeptide and a lipid.

In some embodiments, provided herein are methods of cancer (e.g., in the lung, etc.) by delivery (e.g., via aerosol or gavage) of a liposomal composition comprising a natural or synthetic SOCS polypeptide and a lipid.

In some embodiments, provided herein are methods of treating a pulmonary disorder (e.g., inflammatory lung diseases or conditions, lung cancer, etc.) in a subject comprising administering to the patient an effective dose of a vesicle-encapsulated SOCS polypeptide. In some embodiments, liposome-encapsulated SOCS polypeptides are administered. In some embodiments, vesicle-encapsulated SOCS polypeptide is aerosolized and/or nebulized.

In some embodiments, encapsulated SOCS polypeptides are administered (e.g., to the pulmonary system, to the lungs, via inhalation, etc.) for at least one treatment cycle, wherein: the treatment cycle comprises an administration period of 1 to 75 days (e.g., 1, 2, 3, 4, 5, 6, 7, 8, 9, 10, 12, 14, 16, 18, 20, 25, 30, 35, 40, 45, 50, 55, 60, 65, 70, 75, or any ranges there between), followed by an off period of 1 to 75 days (e.g., 1, 2, 3, 4, 5, 6, 7, 8, 9, 10, 12, 14, 16, 18, 20, 25, 30, 35, 40, 45, 50, 55, 60, 65, 70, 75, or any ranges there between). In some embodiments, amount of SOCS polypeptide in each liposome (e.g., in nanograms) and the volume/number of liposomes administered are balanced to provide a useful therapeutic dose of SOCS polypeptides and sufficient amount of liposomes to contact the treatment area or surface. For example, if a useful therapeutic dose were delivered in too small a number of liposomes, the entire treatment area (e.g., lung surface) would not receive adequate treatment. Likewise, to small of dose of SOCS polypeptide might also be ineffective. In some embodiments, an effective dose of SOCS polypeptide comprises 20 to 5000 mg (e.g., 20 mg, 30 mg, 40 mg, 50 mg, 60 mg, 70 mg, 80 mg, 90 mg, 100 mg, 150 mg, 200 mg, 250 mg, 300 mg, 400 mg, 500 mg, 600 mg, 800 mg, 1000 mg, 1500 mg, 2000 mg, 2500 mg, 3000 mg, 4000 mg, 5000 mg, or any ranges there between) of SOCS polypeptide per administration (e.g., once daily, twice daily, three times daily, four times daily, or more) during the administration period. In some embodiments, the treatment cycle is administered to the patient at least twice. In some embodiments, based on the experiments herein, it is within the expertise of a clinician and/or researcher to determine the appropriate dose of SOCS polypeptide and amount of liposomes for administration to a subject.

In some embodiments, the pulmonary disorder treated by the methods and compositions herein is selected from the group consisting of chronic obstructive pulmonary disease, bronchiectasis, pulmonary infection, cystic fibrosis, alpha-1-antitrypsin enzyme deficiency and a combination thereof. In some embodiments, the methods and compositions herein are useful for the treatment of non-infectious conditions. In some embodiments, the methods and compositions herein are not employed for the treatment of infectious conditions (e.g., bronchiectasis, pulmonary infections, etc.) or other diseases and conditions in which a robust inflammatory response is crucial (e.g., cystic fibrosis). In some embodiments, the methods and compositions herein find use in the treatment of asthma, acute lung injury or adult respiratory distress syndrome, sarcoidosis, hypersensitivity pneumonitis, and inflammatory lung involvement associated with autoimmune conditions such as lupus and rheumatoid arthritis. In some embodiments, the methods and compositions herein are useful for the treatment of cancer (e.g., lung cancer (e.g., small cell lung cancer (SCLC), non-small cell lung cancers (NSCLC) (e.g., squamous cell carcinomas, large cell carcinomas, etc.), bronchial carcinoids, etc.), etc.).

In some embodiments, compositions are administered as a nebulized spray, powder, or aerosol, or by intrathecal administration. In some embodiments, administration comprises inhalation. In some embodiments, compositions are administered intravenously or by another acceptable route of administration.

In some embodiments, compositions are co-administered with one or more additional therapies or pharmaceutical agents. In some embodiments, compositions herein are administered to treat inflammation and one or more additional agents are administered to treat the disease or condition underlying the inflammation or resulting from the inflammation. In some embodiments, the additional agent is an anti-inflammatory agent (e.g., non-steroidal anti-inflammatory drug (NSAID), corticosteroids, etc.). In some embodiments, the additional agent is a treatment (e.g., radiation, surgery, etc.) or pharmaceutical (e.g., chemotherapeutic) for the treatment of cancer.

### EXPERIMENTAL

Experiments were conducted during development of embodiments herein that assess the capacity of products secreted by AMs to attenuate JAK-STAT signaling in AECs. Unexpectedly, it was determined that AMs secrete SOCS1 and SOCS3 proteins in vesicles which can be taken up by AECs to mediate inhibition of cytokine-induced STAT activation. This secretion occurs both in vitro and in vivo, is a tunable phenomenon, and can be dysregulated during inflammation. These findings reveal a previously unappreciated means for intercellular communication in inflammation control.

### Example 1

### Materials and Methods

### Animals

Pathogen-free 125-150 g female Wistar rats from Charles River Laboratories and male C57BL/6 wild-type mice purchased from The Jackson Laboratory were utilized. Animals were treated according to NIH guidelines for the use of experimental animals with the approval of the University of Michigan Committee for the Use and Care of Animals.

### Human Subjects and BAL

Studies were done under a protocol approved by the Institutional Review Board of the VA Ann Arbor Healthcare System and registered as NCT01099410; all subjects gave written informed consent. Flexible fiberoptic bronchoscopy and BAL were performed on 7 healthy volunteer subjects who were never smokers (age 44.4±4.7 years) and 7 healthy current smokers (age 51.1±2.8 years; 20±2.8 pack-years) with no respiratory symptoms or lung function abnormalities. Cell-free BALF was obtained after pelleting macrophages and was stored at −80° C.

### Reagents

RPMI 1640 and F12-K were purchased from Gibco-Invitrogen. PGE2 from Cayman Chemical was dissolved in DMSO and stored under N2 at −80° C. Murine and rat cytokines (IL-6, IFNγ and IL-10) were purchased from Peprotech. Mouse monoclonal Ab against SOCS3 and rabbit polyclonal Ab against SOCS1 were from Abcam and Cell Signaling Technology, respectively. Mouse monoclonal Ab against β actin was from Sigma. FITC-conjugated rabbit polyclonal Abs against SOCS3 and SOCS1 were from Biorbyt. The fluorescent lipid 1-oleoyl-2-{6-[(7-nitro-2-1,3-benzoxadiazol-4-yl)amino]hexanoyl}-sn-glycero-3-phosphocholine (18:1-06:0 NBD PC) was purchased from Avanti Polar Lipids. Rabbit polyclonal Abs against phospho- and total STAT1 and STAT3 were from Cell Signaling Technology. LPS, monensin, hematoxylin and proteinase K were from Sigma. Trypsin enzymatic antigen retrieval solution was from Abcam. Compounds requiring reconstitution were dissolved in PBS, EtOH or DMSO. Required dilutions of all compounds were prepared immediately before use, and equivalent quantities of vehicle were added to the appropriate controls. DMSO or EtOH at the concentrations employed had no direct effect on SOCS3 secretion.

### Macrophage Isolation and Culture

Human AMs were obtained as described herein. Resident AMs and peritoneal macrophages from rats and mice were obtained by lavage of the lung or the peritoneal cavity, respectively. Cells were resuspended in RPMI 1640 to a final concentration of 1-3×10⁶ cells/ml. Cells were allowed to adhere to tissue culture-treated plates for at least 1 h (37° C., 5% CO₂), resulting in >99% of adherent cells identified as macrophages by use of modified Wright-Giemsa stain (Diff-Quick) from American Scientific Products. Rat bone marrow-derived macrophages were obtained from bone marrow cells cultured as described previously (Canetti et al., 2006) for 6 days in 100-mm-diameter Petri dishes in 30% L929 cell supernatant in RPMI 1640 containing 20% FCS, L-glutamine, and penicillin/streptomycin. After 3 days, the cell culture was supplemented with new medium totaling 50% of original volume. Spleens from C57BL/6 mice were minced and passed through a 40 μm filter (BD) to obtain a single cell suspension. Erythrolysis was performed with 10 ml 0.8% ammonium chloride lysis buffer. Subsequently, cells were rinsed with HBSS and PBS/2 mM EDTA/0.5% FCS, followed by incubation with CD16/32 for 15 min at 4° C. to avoid nonspecific binding of antibodies. Cells were subsequently stained with F4/80 antibody for 15 min in 4° C., washed, and flow sorted to high purity (>96%).

### Cell Lines

The following cell lines were obtained from ATCC: 1) rat AEC lines L2 (CCL-149) and RLE-6TN (CRL-2300), spontaneously immortalized lines derived from primary cultures of adult rat AECs; 2) MH-S(CRL-2019), a line derived by SV40 transformation of primary murine AMs; 3) NR8383 (CRL-2192), a line derived by spontaneous transformation of primary rat AMs; 4) normal human adult lung fibroblasts (CCL-210); and 5) U937 cells (CRL-1593), myelomonocytic leukemia cells which were used following differentiation into macrophage-like cells by 100 nM phorbol myristate acetate treatment for 16 h.

RNA Isolation and Quantitative RT-PCR Determination of mRNA Levels of MCP-1

RNA was extracted using Qiagen columns according to manufacturer's instructions and converted to cDNA. MCP-1 mRNA levels were assessed by quantitative (q) RT-PCR performed with a SYBR Green PCR kit (Applied Biosystems) on an ABI Prism 7300 thermocycler (Applied Biosystems). The sequences of the primers used for MCP-1 and β actin amplification, respectively, were: 5′-AGC ATC CAC GTG TTG GCT C-3′ (f) (SEQ ID NO: 1), 5′-CCA GCC TAC TCA TTG GGA TCA T-3′ (r) (SEQ ID NO: 2), and 5′-ACC CTA AGG CCA ACC GTG A-3′ (f) (SEQ ID NO: 3), 5′-CAG AGG CATA CAG GGA CAG CA-3′ (r) (SEQ ID NO: 4). Relative gene expression was determined by the OCT method, and β actin was used as reference gene. Primer efficiency tests were performed on all primers and ranged from 97% to 107%.

### Western Blotting

AMs (3-4×10⁶) were plated in 6-well tissue culture dishes and incubated in the presence or absence of compounds of interest for the indicated amounts of time. Then supernatants were harvested (4 mL) and centrifuged at 500×g (10 min) and 2,500×g (10 min) to yield CM. Secreted proteins were concentrated using 3 kDa Amicon size exclusion filters from Millipore, after an aliquot (150 μL) was kept for LDH assay. Protein concentrations were determined by the DC protein assay (modified Lowry protein assay) from Bio-Rad. Samples containing 30 μg protein were separated by SDS-PAGE using 12% gels and then transferred overnight to nitrocellulose membranes. After blocking with 4% BSA, membranes were probed overnight with commercially available Abs directed against SOCS (titer of 1:500), phospho-STAT and total STAT (titer of 1:1000), and β actin (titer of 1:10,000). Following incubation with peroxidase-conjugated goat anti-rabbit (or anti-mouse) secondary Ab (titer of 1:10,000) from Cell Signaling Technology, film was developed using ECL detection from Amersham Biosciences. Relative band densities were determined by densitometric analysis using NIH Image J software.

### Detection of SOCS3 by ELISA

A commercially available ELISA kit (Cloud-Clone) was utilized to quantify SOCS3 levels in AEC lysates or in BALF sonicated (Branson Sonifier 250; 40% duty cycle, output 3) for 10 sec on ice x 3 to disrupt MPs.

### Detection of TNF by ELISA

TNF was measured in the cell culture supernatant from AMs plated in 96-well plates at a density of 0.5×10⁶ cells/100 μL. Supernatants were collected after 1 h, cell debris was removed by centrifugation (500 g, 10 min), and samples analyzed by immunoassay kits from R&D systems.

### Cytotoxicity

Leakage of cytosolic proteins was assessed by cytotoxicity detection kit (LDH) from Roche Diagnostics. AMs were cultured and supernatants were centrifuged for 10 min at 500 g and 2500 g, and then LDH release assay was performed.

### Purification of MPs and Exos

Rat AMs were cultured and the culture supernatant was harvested for the enrichment of MPs (Brogan et al., 2004; herein incorporated by reference in its entirety) and Exos (Thery et al., 2006: herein incorporated by reference in its entirety). CM obtained from AM supernatants as described above was centrifuged at 17,000×g for 160 min. The final pellets were resuspended in 200 μL of Ca²⁺-free Tyrode's Buffer for flow cytometric analysis or resuspended in RPMI 1640 for in vitro studies or PBS for in vivo studies, while the remaining supernatants were further enriched for Exos by ultracentrifugation at 100,000×g at 4° C. for 90 min.

### Flow Cytometry Analysis

Flow cytometry was performed using a Becton Dickinson FACS Canto 2. MPs were incubated with annexin V-FITC or annexin V-phycoerythrin control from BD PharMingen for 20 min at room temperature in dark. Then samples were permeabilized with 0.2% NP40 and incubated with 0.5 μg of FITC-conjugated SOCS3 Ab. The light scatter and fluorescence channels were set at logarithmic gain. Calibration of MP size was performed using a Polybead Sampler Kit from Polysciences, Inc. Samples were immediately analyzed with flow cytometry. Using 1.0 μm beads as standard, we quantified the number of MPs in known volumes of the MP aliquot. Ten thousand events were acquired for each sample. For MP quantification, up to 25,000 events were acquired. Data were analyzed using FlowJo software (Becton Dickinson).

### AM and MP Staining and Microscopy

To label plasma membranes, AMs were incubated with 100 μM of the fluorescent lipid 18:1-06:0 NBD PC for 20 min on ice in the dark, and then washed 3 times before plating them. Slides were mounted in SlowFade Gold antifade mounting media with 4,6 diamidino-2-phenylindole (DAPI) (Molecular Probes) to visualize nuclei. Cells were imaged on a Nikon Eclipse E600 Microscope (magnification 100×).

For MPs, rat AMs were cultured in RPMI without Phenol Red, and then AM supernatant was harvested and processed for the enrichment of MPs. MPs were incubated with annexin V-FITC from BD Pharmingen for 20 min at room temperature in the dark and were imaged on a Nikon TE300 with a 60× oil immersion objective (NA 1.40, total magnification of 600×).

### RNA Interference

RNA interference was performed according to a protocol provided by Dharmacon. Rat AMs were transfected using lipofectamine RNAiMax reagent from Invitrogen with 100 nM non-targeting SMARTpool control or specific ON-TARGET SMARTpool SOCS3 (SOCS3) siRNA from Dharmacon. After 72 h of transfection, AMs were washed and incubated for 48 h with RPMI 1640.

### In Vitro Transfer Experiments

To assess the uptake and functional effects of secretory products of rat AMs in recipient rat AECs, AECs were incubated with F12-K medium or CM, at either 37 or 4° C. for times ranging from 30 min to 2 h. Alternatively, they were incubated with either MPs or Exos isolated from AM-derived CM, or with CM that had been depleted of MPs by centrifugation. SOCS3 transfer was determined following a 2-h incubation with AM-derived CM by quantifying immunoreactive SOCS3 in AEC lysates using ELISA. Uptake of MPs was determined by labeling MPs with annexin V-FITC, incubating them with AECs for 1 hat a ratio of 10:1, and determining fluorescence in AECs by flow cytometry after trypsinization and washing. To evaluate modulation of STAT activation AECs were pretreated with CM, MP-depleted CM, MPs, or Exos prior to treatment with IL-6 (20 ng/ml) or IFNγ (5 ng/ml) for 1 h. Inhibition of IL-6-induced STAT3 and IFNγ-induced STAT1 activation was assessed by WB using Abs directed against Tyr705 phospho-STAT3 and Tyr701 phospho-STAT1, respectively. The contribution of SOCS3 to inhibition of IL-6-induced STAT3 or IFNγ-induced STAT1 activation was determined by comparing the inhibitory ability of CM obtained from AMs pretreated for 3 d with SOCS3 vs. control siRNA. SOCS3 knockdown in cell lysates and CM was evaluated by WB.

### Mouse Model of Cigarette Smoke Exposure

8-10 wk-old female C57BL/6 mice were exposed for 2 h/day for 3 or 7 d to mainstream cigarette smoke from research cigarettes, as described (Phipps et al., 2010; herein incorporated by reference in its entirety); control mice were unexposed. BALF was obtained following sacrifice and analyzed for SOCS1 and SOCS3 content by WB.

### In Vivo Experiments

Levels of SOCS3 and SOCS1 in concentrated BALF from naïve or smoked mice or healthy human never smokers and current smokers were determined by WB and/or ELISA. To evaluate the ability of immunomodulatory substances to influence BALF levels of SOCS3, mice were subjected to oropharyngeal administration into the lungs of 50 μl of saline containing 15 μg PGE2 and/or LPS, or vehicle alone. BALF was harvested 3 h later and analyzed by WB for SOCS3. For in vivo transfer experiments, MPs from rat AMs and peritoneal macrophages were isolated, quantified using flow cytometry, and 3×10⁶ MPs were oropharyngeally administered per mouse. Two h later, IFNγ (0.1 μg) was administered by the same route. Responses analyzed 1 h thereafter in lung homogenates following initial lung lavage to remove AMs included Tyr701 phospho-STAT1 and Tyr705 phospho-STAT3 by WB, MCP-1 mRNA determination by qRT-PCR, and immunostaining.

### Immunohistochemical Staining and Image Analysis of Lung Sections

Lungs were harvested from mice treated as described herein, fixed in formalin and processed (Brock et al., 2001; herein incorporated by reference in its entirety). A trypsin enzymatic antigen retrieval solution was applied for 15 min at room temperature. Rabbit polyclonal antibodies against phospho-STAT1 (titer 1:50) were applied overnight at 4° C. Nuclei were briefly counterstained with hematoxylin after completion of immunostaining. Images were taken using a Nikon Eclipse E600 Microscope (magnification 40×). p-STAT1 staining was quantified by first separating the colors using color deconvolution plugin (Image J software) and performing densitometric analysis of red staining in 10 randomly-selected fields, which was expressed relative to the area of the whole field.

### Example 2

### SOCS1 and SOCS3 Secretion in the Lung

### SOCS3 Protein Mediates Inhibition of AEC STAT Activation by AM-Derived Conditioned Medium (CM)

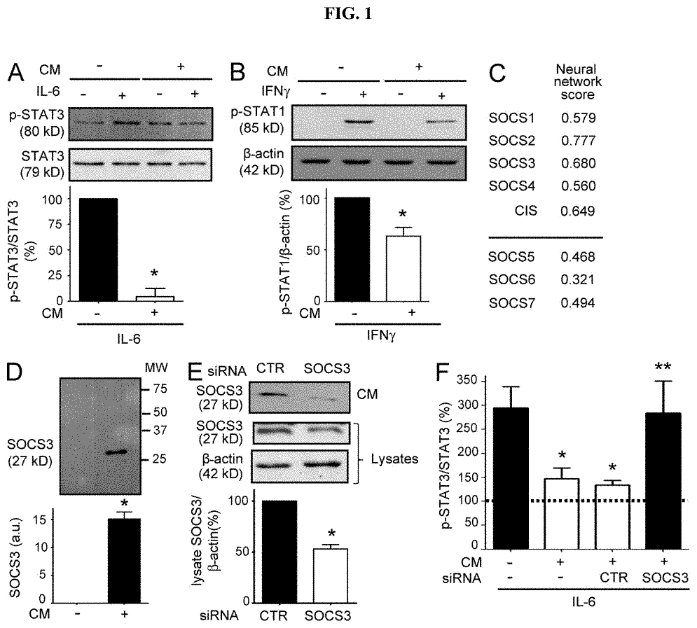

CM was collected from primary rat AMs that had been adhered, cultured overnight and centrifuged at 500×g (to remove floating cells) and 2500×g (to remove debris and apoptotic bodies). To assess its immunoregulatory capacity, CM was added to rat L2 AECs 2 h before addition of pro-inflammatory cytokines. As compared to RPMI 1640 alone, AM-derived CM inhibited IL-6-induced activation of STAT3 (indicated by phosphorylation on Tyr 705) (FIG. 1A) as well as IFNγ-induced activation of STAT1 (indicated by phosphorylation on Tyr 701) (FIG. 1B); these effects were confirmed using RLE-6TN, another non-transformed rat AEC line (not shown). To address the possibility that this inhibition of STAT activation might be attributable to increased expression of endogenous SOCS protein in response to treatment with the cytokine itself, the effect of one hour incubation with IL-6 on levels of SOCS3 protein was determined by Western blot (WB) analysis in lysates of AECs. These data demonstrated no meaningful increase in endogenous SOCS3 protein expression within this short time frame, instead pointing to the actions of an inhibitory molecule in AM CM.

Experiments were conducted during development of embodiments here in to assess whether the inhibitor of STAT activation in AM-derived CM is a SOCS protein. Although members of the SOCS family have never previously been identified extracellularly, informatics analysis supported the plausibility of SOCS secretion. While SOCS1 and SOCS3 lack an N-terminal leader sequence typical of proteins secreted via conventional ER-Golgi pathways (Bendtsen et al., 2004b; herein incorporated by reference in its entirety), both are among those SOCS family members meeting prediction criteria (SecretomeP 2.0-derived neural network score >0.5) (Bendtsen et al., 2004a; herein incorporated by reference in its entirety) for secretion by unconventional pathways (FIG. 1C)—a phenomenon now well-recognized for “leaderless” proteins (Nickel and Rabouille, 2009; herein incorporated by reference in its entirety). In view of its higher neural network score, the presence of SOCS3 in concentrated AM-derived CM was assessed by performing WB analysis. This revealed a single band at the expected molecular weight for SOCS3 (FIG. 1D).

To confirm the identity of this band as the product of the SOCS3 gene, it was verified that its level declined substantially in CM obtained from AMs treated with SOCS3 siRNA as compared to that obtained from AMs treated with control scrambled siRNA (FIG. 1E). The ability of CM from AMs in which SOCS3 expression had been knocked down to inhibit activation of STATs was then assessed. CM from AMs treated with SOCS3 siRNA, but not control siRNA, lost its ability to inhibit AEC STAT3 activation in response to IL-6 (FIG. 1F) as well as STAT1 activation in response to IFNγ (not shown). Although STAT activation is also negatively regulated by tyrosine phosphatases SHP1 and SHP2, these phosphatases are not predicted by SecretomeP 2.0 to be unconventionally secreted.

### SOCS3 Secretion by AMs Proceeds Via an Unconventional Vesicular Pathway Mainly Involving MPs

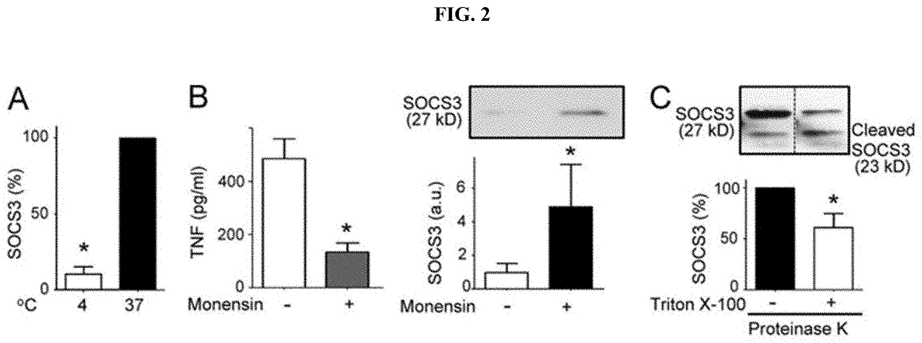

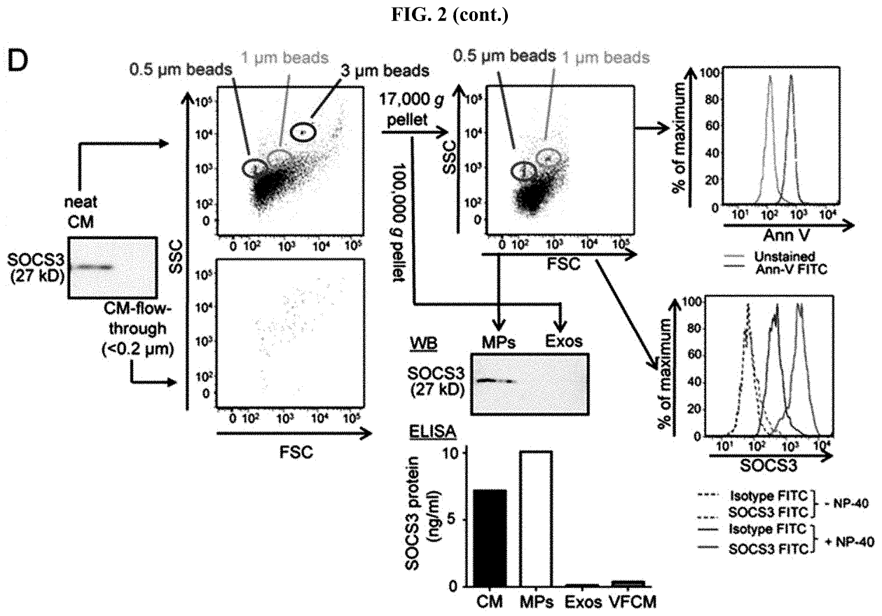

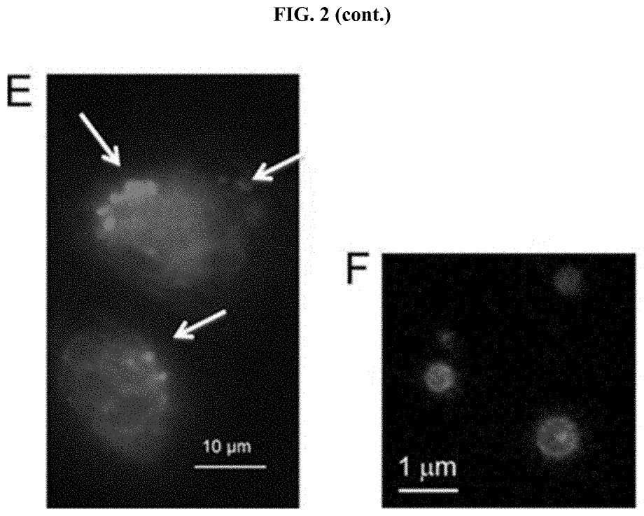

SOCS3 secretion was found to be unassociated with LDH release, indicating it is not a manifestation of cytotoxicity. In addition, it was markedly reduced at 4° C., indicating that it is an energy-dependent phenomenon (FIG. 2A). To confirm that SOCS3 is indeed released by AMs through unconventional secretion, the effects of monensin, an inhibitor of conventional secretion, were tested. Monensin inhibited rat AM secretion of the known conventionally secreted protein TNF (FIG. 2B, left); by contrast, it increased secretion of SOCS3 (FIG. 2B, right), as it has previously been recognized to do for other unconventionally secreted proteins (Rubartelli et al., 1990; herein incorporated by reference in its entirety). Similar results were obtained using brefeldin A, another inhibitor of conventional secretion. Unconventional secretion can be vesicular in nature; the finding that SOCS3 in AM-derived CM was more sensitive to proteolysis in the presence of a detergent (FIG. 2C) implied its packaging within a membranous structure, such as an extracellular vesicle.

The two main types of extracellular vesicles capable of harboring protein cargo are microparticles (MPs) and exosomes (Exos). MPs originate by budding or shedding from the plasma membrane, are between 0.1-1 μm in diameter, and are annexin V-positive owing to the phosphatidylserine (PS) on their outer surface (Hugel et al., 2005; herein incorporated by reference in its entirety). By contrast, Exos originate from endosomal membranes and are <0.1 μm in diameter. To better characterize the type of vesicles containing SOCS3, AM CM was passed through a 0.2 μm filter, which separates MPs in the filtrate from Exos contained in the flow-through. The neat CM was verified to contain SOCS3 (by WB) as well as MPs, as indicated by flow cytometric demonstration of a population of particles with a diameter of 0.5-1 μm that were largely annexin V-positive, whereas the flow-through contained neither SOCS3 nor MPs (FIG. 2D). MPs budding from AMs were visualized directly by fluorescence microscopy after labeling the plasma membranes of cells in suspension with the fluorescent lipid 18:1-06:0 NBD PC prior to plating (FIG. 2E). To confirm that SOCS3 is in MPs, they were isolated from CM by centrifugation at 17,000×g (Brogan et al., 2004; herein incorporated by reference in its entirety). The presence of MPs in this pellet was verified by visualizing annexin V-positive vesicles of varying sizes by fluorescence microscopy (FIG. 2F), and this MP fraction also contained SOCS3 protein, as determined by WB analysis and by a commercially available ELISA (FIG. 2D). The presence of SOCS3 within these MPs was further confirmed by their flow cytometric positivity when stained with a fluorochrome-conjugated anti-SOCS3 Ab (different from that employed for WB analysis) with, but not without, membrane permeabilization by gentle detergent treatment using NP-40 (FIG. 2D). Exos, pelleted by 100,000×g centrifugation of the 17,000×g supernatant, contained no SOCS3, as determined by either WB or by ELISA; ELISA also verified the absence of SOCS3 in CM depleted of both types of vesicles (FIG. 2D).

### Uptake of SOCS3-Containing MPs by AECs Inhibits Target Cell STAT3 Activation

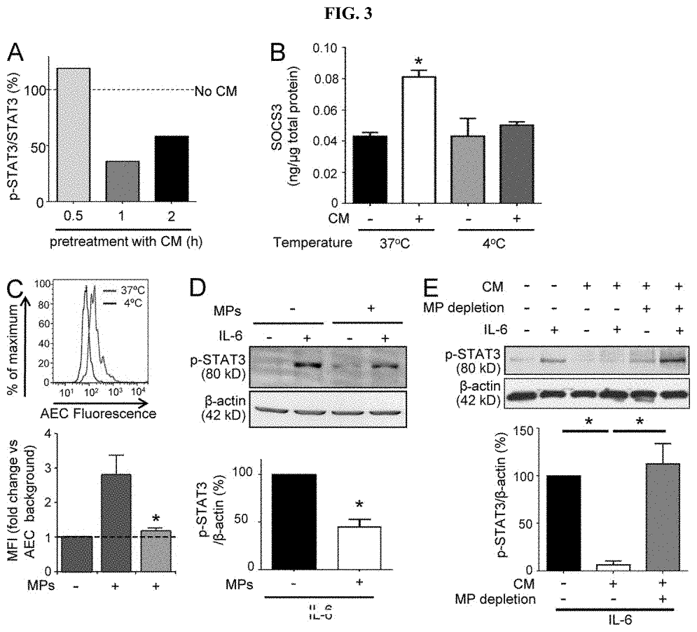

Experiments were conducted during development of embodiments herein to test whether the ability of vesicles to be internalized via either membrane fusion or endocytosis (Mause and Weber, 2010; herein incorporated by reference in its entirety) facilitates the anti-inflammatory actions in target AECs of SOCS-containing vesicles released by AMs. Indeed, the duration of AEC pretreatment with AM CM required to attenuate subsequent STAT3 activation (>30 min, maximal by 60 min) (FIG. 3A) is consistent with the time frame that has been previously established for vesicular uptake (Sadallah et al., 2008; herein incorporated by reference in its entirety). To directly evaluate the uptake of AM-derived SOCS3 by AECs, ELISA was used to quantify intracellular levels of SOCS3 in lysates of AECs prepared before and after a 2-h incubation with AM CM. Baseline intracellular SOCS3 levels doubled following incubation with CM at 37° C. but remained unchanged following incubation at 4° C. (FIG. 3B). In parallel fashion, AECs incubated at 37° C. with FITC-annexin V-labeled AM-derived MPs exhibited an increase in fluorescence as determined by flow cytometry, whereas incubation at 4° C. resulted in no such increase (FIG. 3C). Together these data demonstrate energy-dependent uptake by AECs of AM-derived MPs as well as SOCS3. Since SOCS3 was enriched within AM-derived MPs and these MPs could be taken up by AECs, the ability of purified MPs to reproduce the anti-inflammatory actions of neat AM CM on AECs was evaluated. MPs, added at a commonly employed ratio of 10 MPs:1 target cell (Gasser et al., 2003; herein incorporated by reference in its entirety), were indeed capable of inhibiting IL-6-induced STAT3 activation in AECs (FIG. 3D). In reciprocal fashion, AM CM lost its ability to inhibit AEC STAT3 activation following depletion of MPs by centrifugation at 17,000×g (FIG. 3E).

### SOCS1 Protein is Secreted in Exos and Exerts Inhibitory Effects on AEC STAT1 Activation

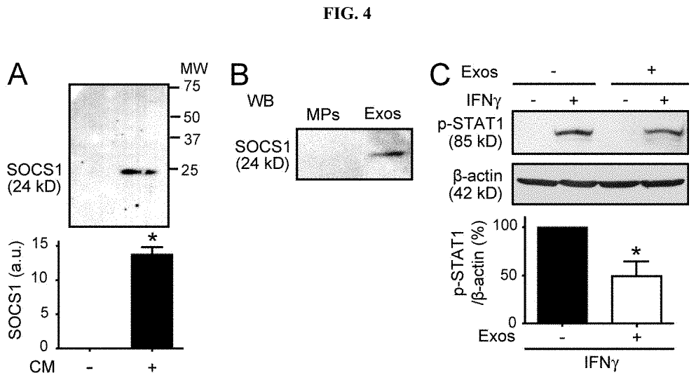

Because SOCS1 was also predicted to be secreted (FIG. 1C), we evaluated its presence in AM CM using WB. It too was identified as a single band at the appropriate molecular weight (FIG. 4A). However, differential centrifugation revealed SOCS1 to be present primarily in the Exos fraction (pellet obtained from 100,000×g centrifugation of the 17,000×g supernatant) (FIG. 4B), rather than in the MP fraction as was the case with SOCS3 (FIG. 2D). Consistent with this finding, flow cytometric staining of MPs using FITC-conjugated anti-SOCS1 following gentle detergent permeabilization was negative. As shown for MPs (FIG. 3D), the functional activity of AM-derived Exos was confirmed by their ability to inhibit IFNγ-induced STAT1 activation in AECs (FIG. 4C). Moreover, the ability of AM CM to inhibit IFNγ-induced STAT1 activation was attenuated by pretreatment of AMs with SOCS1 siRNA. Together, these data indicate that SOCS1 contained in Exos abrogates STAT1 activation.

### SOCS3 Secretion is a Regulated Phenomenon In Vitro

Macrophage adherence to plastic culture dishes is recognized to trigger a burst of activation (Kelley et al., 1987; herein incorporated by reference in its entirety). It was found that adherence resulted in a rapid burst of release of both SOCS3 (FIG. 5A, top) as well as MPs (quantified by flow cytometry) (FIG. 5A, bottom), followed by a much lower basal rate of secretion post-adherence. SOCS3 secretion increased as early as 5 min after AM adherence (FIG. 5B). The rapidity of this response is consistent with the known kinetics of MP release described for monocytes (MacKenzie et al., 2001; herein incorporated by reference in its entirety). Experiments were conducted to determine if AM secretion of SOCS proteins could be regulated by known immunomodulatory molecules. The lipid mediator PGE2 down-regulates many features of AM activation (Aronoff et al., 2004; Bourdonnay et al., 2012; herein incorporated by reference in their entireties), and the cytokine IL-10 is well-known for its anti-inflammatory and immunosuppressive actions (Sabat et al., 2010; herein incorporated by reference in its entirety); these are both are known to be secreted by AECs (Chauncey et al., 1988; Jose et al., 2009; herein incorporated by reference in their entireties). Both rapidly potentiated basal secretion of SOCS3 when added during the post-adherence phase (FIGS. 5C and D), and PGE2 also increased secretion of SOCS1. By contrast, the pro-inflammatory endotoxin lipopolysaccharide (LPS) decreased basal SOCS3 secretion in AMs (FIG. 5D). Unlike the effects of cell adherence (FIG. 5A), the ability of these immunomodulatory substances to rapidly increase (IL-10 and PGE2) or decrease (LPS) SOCS3 secretion by cultured AMs was unassociated with changes in the number of MPs secreted (FIG. 5E, left), indicating instead an alteration in the content of SOCS packaged per MP (FIG. 5E, right).

### Expression and Secretion of SOCS3 by Various Cell Populations

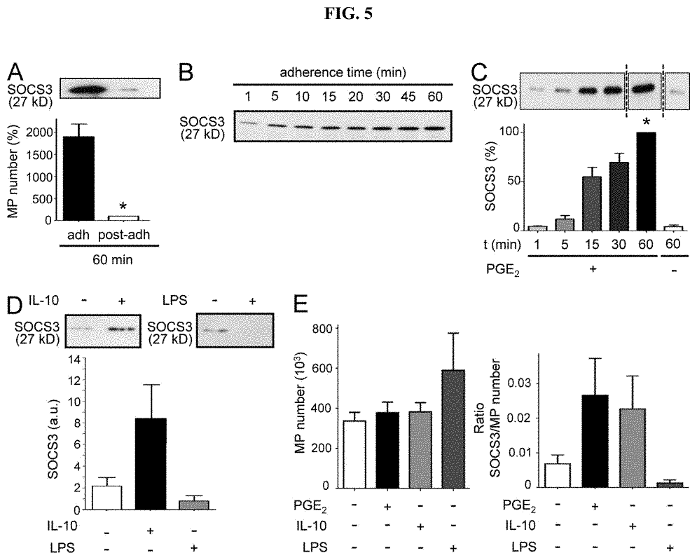

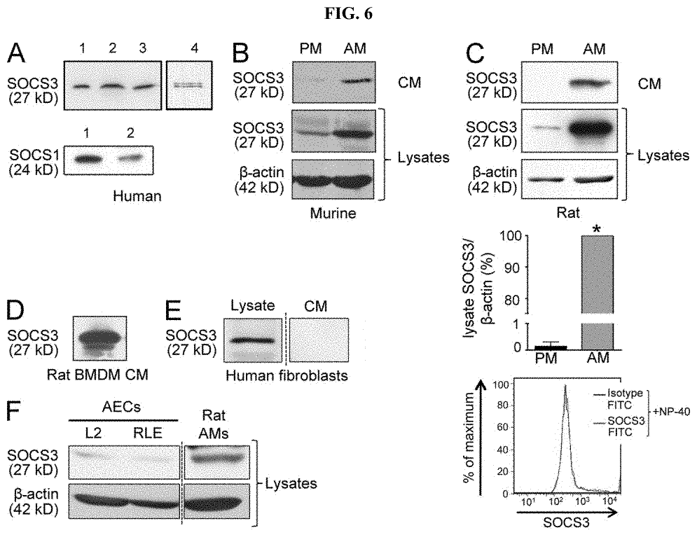

As described for rat AMs, it was also demonstrated that robust secretion of SOCS3 and SOCS1 proteins by resident AMs obtained from healthy human subjects (FIG. 6A) as well as SOCS3 from mouse AMs (FIG. 6B, top). Secretion of both SOCS proteins was similarly observed in cell lines derived from primary rat (NR8383 line) and mouse (MH-S line) AMs. By contrast, analysis of CM derived from cultured peritoneal macrophages from mice (FIG. 6B, top) and rats (FIG. 6C, left, top) revealed no appreciable SOCS3, and no SOCS3 was identified by flow cytometry within permeabilized MPs isolated from rat peritoneal macrophage-derived CM (FIG. 6C, right); notably, they also expressed very little intracellular SOCS3 (FIG. 6B, bottom and 6C, left, bottom). Macrophages isolated from mouse spleen as well as phorbol ester-differentiated U937 human monocyte-like cells likewise exhibited minimal degrees of SOCS3 secretion and expression (not shown). However, SOCS3 was expressed and secreted by rat bone marrow-derived macrophages (FIG. 6D), implying that this phenomenon is not limited to the lungs. In contrast to the apparent correlation between expression and secretion observed in macrophages (FIGS. 6B and C), normal human lung fibroblasts expressed abundant levels of SOCS3 but failed to secrete it (FIG. 6E). These data show that abundant intracellular expression of SOCS proteins is necessary but not sufficient for their secretion, and confirm that secretion of SOCS proteins is an independently regulated event, consistent with the findings in FIG. 5. Notably, AECs themselves expressed negligible levels of intracellular SOCS3 protein (FIG. 6F), indicating the possible importance of them acquiring biologically active SOCS3, such as from donor AMs instead.

### Effects of AM-Derived SOCS3 on Pulmonary STAT Activation In Vivo

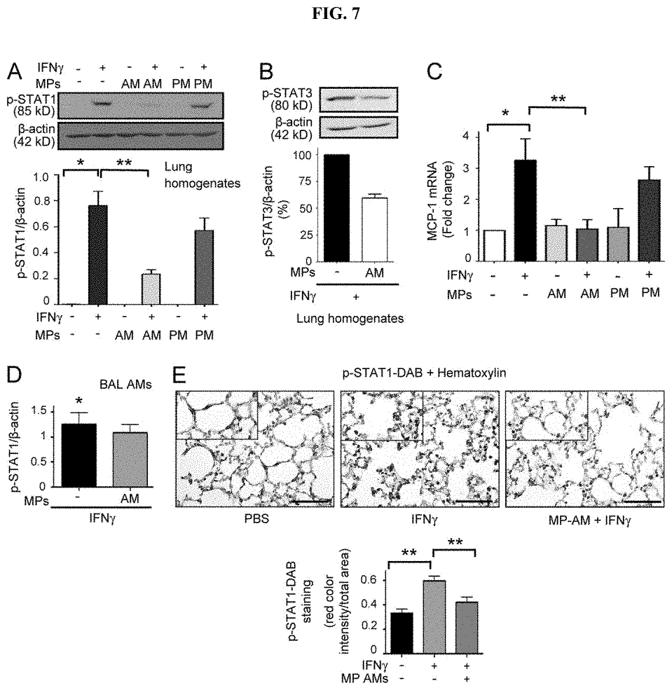

The in vivo ability of AM-derived SOCS3 to influence pulmonary inflammatory signaling by the direct intrapulmonary administration of MPs was tested employing as negative controls MPs that lacked SOCS3. SOCS3 protein exhibits 100% homology between rat and mouse; experiments utilized rat AMs as a source of MPs and normal C57BL/6 mice as recipients. IFNγ activates not only STAT1 but also STAT3 (Qing and Stark, 2004; herein incorporated by reference in its entirety). Intrapulmonary pretreatment with ˜3×10⁶ MPs/mouse inhibited IFNγ-induced STAT1 activation (FIG. 7A), STAT3 activation (FIG. 7B), and mRNA expression of the STAT-dependent chemokine, monocyte chemotactic protein 1 (MCP-1, or CCL2) (FIG. 7C) in lung homogenates depleted of AMs by lavage just prior to harvest. No corresponding inhibition of STAT1 activation was detected in the lavaged AMs themselves (FIG. 7D), indicating that AECs were the target cells responsible for the inhibition noted in lung homogenates. That phosphorylated STAT1 was found mainly in AECs of the IFNγ-challenged lung and that this AEC STAT1 activation was attenuated by prior intrapulmonary administration of AM-derived MPs was verified by immunohistochemical staining of lung sections for phospho-STAT1 (FIG. 7E). In contrast to the effects of AM-derived MPs, administration of the same number of rat peritoneal macrophage-derived MPs, isolated from CM which lacks SOCS3 (FIG. 6C), failed to attenuate lung STAT1 activation (FIG. 7A) and MCP-1 mRNA expression (FIG. 7C). These negative data for PS-positive but SOCS3-negative peritoneal macrophage-derived MPs exclude the possibility that the anti-inflammatory effects of AM-derived MPs can be explained by potential nonspecific anti-inflammatory effects attributable to the PS on their surface.

### Regulation and Dysregulation of SOCS Secretion in the Lung In Vivo

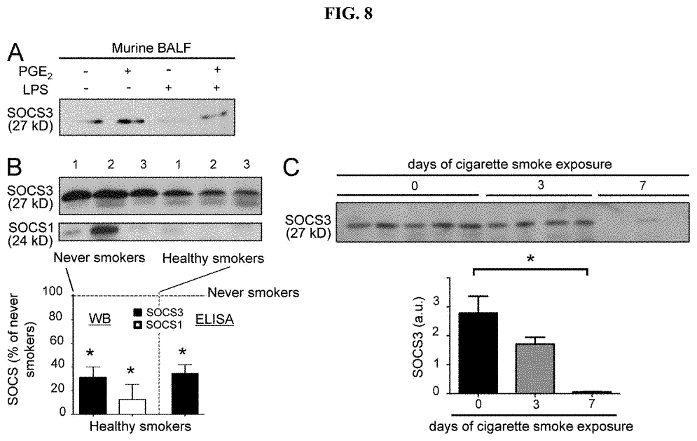

Experiments were conducted to determine whether SOCS secretion occurred in the lung in vivo and whether it was a regulated phenomenon as was observed in vitro. SOCS3 was readily identified by WB in concentrated bronchoalveolar lavage fluid (BALF) obtained from the lungs of individual naïve mice (FIG. 8A). Quantitation of SOCS3 in sonicated BALF from naïve mice by ELISA yielded a level of 10.38±0.96 ng/ml—a concentration which substantially exceeds that reported for most cytokines. Furthermore, just as was observed in vitro, the level of SOCS3 in BALF increased and decreased 3 h following intrapulmonary administration of PGE2 and LPS, respectively, and an intermediate level was observed when they were co-administered (FIG. 8A).

As demonstrated in BALF from naïve mice (FIG. 8A), SOCS3 as well as SOCS1 was readily identified by WB in BALF obtained by fiberoptic bronchoscopy from healthy, never-smoking human volunteers (FIG. 8B), consistent with their ex vivo secretion by cultured AMs from these same subjects (FIG. 6A). It has long been recognized that a chronic state of pulmonary inflammation is elicited by cigarette smoking which precedes the development of smoking-associated lung disease (Cosio et al., 2009; Holt, 1987; herein incorporated by reference in their entireties). Therefore levels of both SOCS3 (by WB and ELISA) and SOCS1 (by WB) in BALF were evaluated from a cohort of never smokers and a cohort of current smokers (20±2.8 pack-years) without respiratory symptoms or lung function abnormalities. By WB analysis in a subset of 4 subjects per group, levels of both SOCS3 and SOCS1 were significantly decreased by 65% and 85%, respectively, in the current smokers as compared to the never smokers. Moreover, iBALF SOCS3 levels as determined by ELISA were significantly and similarly reduced by ˜65% in the entire group of current smokers as compared to the never smokers (FIG. 8B). Typical features of cigarette smoking-associated inflammation are seen in mice after just a few days of cigarette smoke exposure (John et al., 2014; herein incorporated by reference in its entirety). We exposed C57BL/6 mice to mainstream cigarette smoke from standardized research cigarettes as previously described (Phipps et al., 2010; herein incorporated by reference in its entirety) for 2 h/d for either 3 or 7 d. As compared to smoke-unexposed mice, smoke-exposed mice demonstrated a time-dependent decline in SOCS3 levels in BALF which was dramatic by day 7 (FIG. 8C). Together, these data in humans and mice document a substantial impairment in the in vivo secretion of SOCS proteins in the alveolar space in association with the known inflammatory response that characterizes cigarette smoke exposure.

### Example 3

### Liposome Encapsulation and Delivery

Although individual SOCS isoforms are classically thought to inhibit different STAT isoforms (e.g., SOCS1 inhibits STAT1, SOCS3 inhibits STAT3), substantial overlap exists in the STAT target specificity of particular SOCS isoforms. SOCS1 and SOCS3 are targeted herein for liposome encapsulation. Although experiments conducted during development of embodiments herein demonstrate that alveolar macrophages selectively packaged natural SOCS1 within exosomes (˜40-100 nm diameter) and SOCS3 within microparticles (˜100-1000 nm) for secretion, a range of liposome sizes is used as vehicles for delivery of both isoforms of SOCS.

His-tagged SOCS1 and SOCS3 are each cloned into a bacterial expression vector and expression is induced in E. coli by isopropyl-beta-D-thiogalactopyranoside. The expressed protein is recovered on a Ni-NTA column, His tag is removed with TEV protease, and it is purified by FPLC. Purity is verified, for example, by gel electrophoresis and Coomassie staining.

Lipid films are generated by mixing, for example, dioleoylphosphocholine and dioleoylphosphoglycerol in a 1:1 molar ratio. Liposomes are formed by hydrating the lipid films with purified recombinant SOCS protein in PBS (or PBS alone for empty liposomes). Extrusion through either 70 or 400 nm membrane filters yields unilamellar liposomes, which are purified by ultracentrifugation. Liposomes of 480 Â±6.5 nm diameter (by dynamic light scattering) with a surface charge (by zeta potential analyzer) of −27.0 Â±1.27 mV, and a protein content (by HPLC) of 4.5 Âμm per 1.26 Âμmoles of lipids have been generated. Liposomes too are subjected to electrophoresis to verify purity and integrity of the encapsulated recombinant SOCS protein.

Efficacy of SOCS-containing liposomes is initially established by demonstrating their ability to attenuate STAT phosphorylation—a measure of its activation—in cells or lungs that have been challenged with cytokine to activate the JAK-STAT pathway.

### Example 4

### Lung Cancer

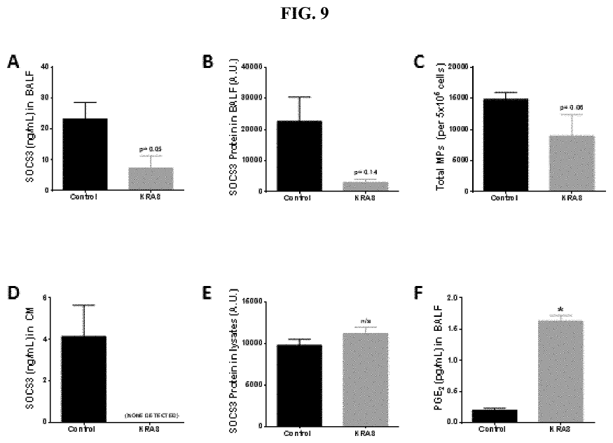

KrasLSL-G12D mice were administered adenoviral Cre recombinase via intra-tracheal instillation. After 16 weeks, the mice were sacrificed and bronchoalveolar lavage fluid (BALF) was collected. Alveolar macrophages (AMs) were isolated from the BALF and were cultured in serum-free RPMI for 24 hours. The resulting AM conditioned medium (AM-CM) was collected. Cell-free BALF and AM-CM were depleted of apoptotic bodies and were concentrated using 3 kDA exclusion filters. SOCS3 and PGE2 levels were analyzed by ELISA. Microparticle (MP) numbers were obtained from AM-CM by means of Annexin-V staining and flow cytometry, using 3 um labeled beads as a standard (FIG. 9A-F). These experiments demonstrate that SOCS3 secretion is decreased in both BALF and AM-CM from mice with KRAS lung cancer.

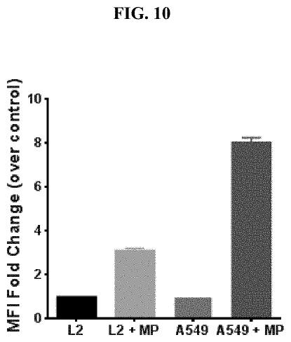

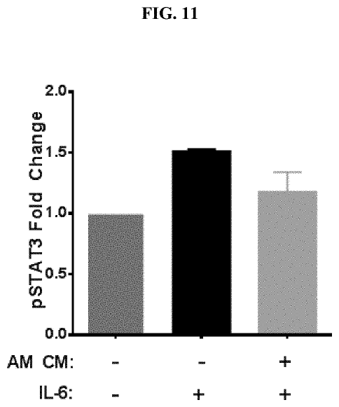

Experiments conducted during development of embodiments herein demonstrate that AM MP uptake is enhanced in A549 adenocarcinoma cells compared to normal AECs, and they exhibit inhibition of IL-6 induced STAT3 activation when exposed to AM-CM (FIGS. 10 and 11).

### Example 5

### Vesicle-Encapsulated SOCS for Inflammatory Lung Disease and Lung Cancer

### Methods

### Liposomes

Recombinant human SOCS3 was expressed in bacteria and purified to yield a single band on SDS-PAGE at the expected molecular size. Varying amounts of purified SOCS protein was mixed with phosphatidylcholine and extruded through mesh to yield liposomes of ˜50 and 400 nm in diameter. SOCS3 in liposomes was verified by Western blot analysis using a SOCS3 antibody. Control liposomes consist of empty phosphatidylcholine vesicles lacking any encapsulated protein.

### In Vitro Experiments

A volume of SOCS3-containing liposomes containing ˜2-20 ng protein (selected to approximate the amount of SOCS protein contained in natural macrophage-derived vesicles which were determined to inhibit signaling in recipient epithelial cells) was added to culture wells of adherent respiratory epithelial cells. Control was addition of an equivalent volume and number of empty liposomes. Three types of respiratory epithelial cells were employed. To represent normal alveolar epithelial cells, rat L2 cells were used. To represent normal bronchial/airway epithelial cells, human BEAS-2b cells were used. To represent malignant epithelial cells, human A549 adenocarcinoma cells were used. Liposomes were incubated with epithelial cells for 2 hours, after which they were removed and cells were washed. Thereafter, biological responses in the epithelial cells were stimulated by addition of various cytokines, selected to activate particular signaling pathways and transcription factors. These include interferon-gamma (IFN-γ) to activate STAT1; interleukin-6 (IL-6) to activate STAT3; and IL-4/IL-13 to activate STATE, STAT3, and NF-κB. At various times after cytokine addition, cells were harvested for analysis of: 1) phosphorylation of STAT (or other transcription factors); 2) chemokine or cytokine mRNA by RT-PCR or protein by ELISA; 3) proliferation (by CyQuant DNA dye-binding); and 4) apoptosis (by annexin-V staining via flow cytometry).

### In Vivo Experiments

A volume of SOCS3-containing liposomes containing ˜35 ng of protein (selected to approximate an amount of SOCS protein contained in natural macrophage-derived vesicles where were determined to inhibit signaling in the lung in vivo) was administered to the lungs of normal C57BL/6 mice via the oropharyngeal route. 2 hours later, IFN-γ was administered by the same route to stimulate STAT activation. 1 hour thereafter, lungs were harvested and homogenates subjected to analysis of STAT phosphorylation (by Western blot) and mRNA expression (by RT-PCR) for the IFN-γ-inducible gene, IP-10.

### Results

### In Vitro Effects of SOCS3-Containing Liposomes on Signaling Responses in Epithelial Cells

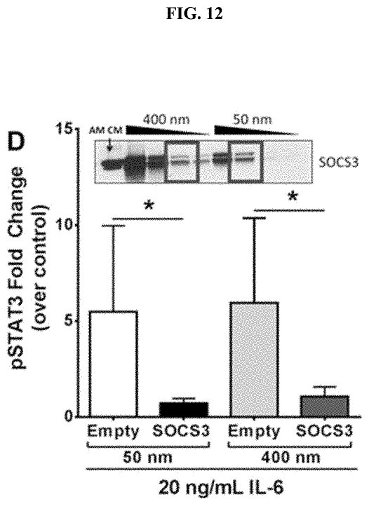

Serial dilutions of SOCS3-containing liposomes of both sizes were loaded onto a SDS-PAGE gel. For comparison, conditioned medium from cultured rat alveolar macrophages (which contain SOCS3 in microvesicles) was also loaded. Proteins were electrophoresed, transferred to a membrane, and probed with an antibody to SOCS3. As seen in FIG. 12 (top), a doublet band of the appropriate molecular size (˜27 kDa) recognized by the SOCS3 antibody was present in the macrophage conditioned medium. Serial dilutions of both sized liposomes also showed doublet bands at the expected molecular size recognized by the antibody. The boxes in FIG. 12 (top) indicate the dilutions that were used in a series of experiments testing effects on STAT3 phosphorylation in L2 normal rat alveolar epithelial cells in response to IL-6. Cells pretreated with empty liposomes showed a robust increase in STAT3 phosphorylation following IL-6 stimulation (˜5-6-fold higher than that observed with no IL-6 stimulation). Pretreatment with both sizes of SOCS3-containing liposomes resulted in a statistically significant ˜80-90% inhibition of IL-6-stimulated STAT3 phosphorylation as compared to that observed with empty liposome pretreatment (FIG. 12, bottom).

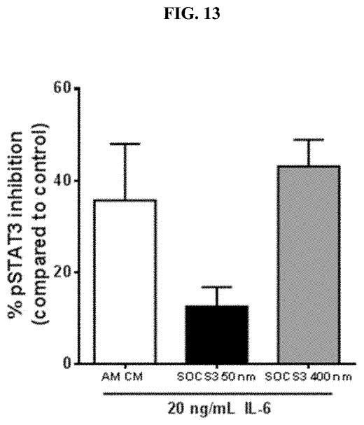

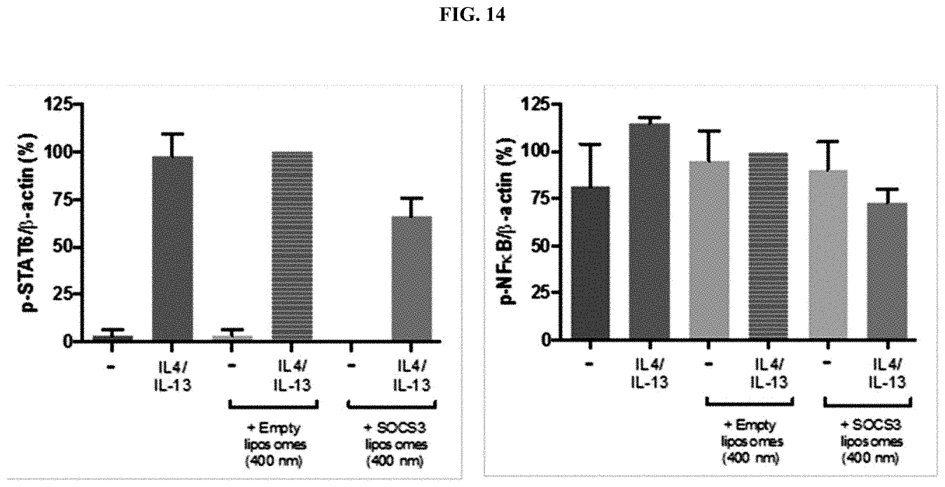

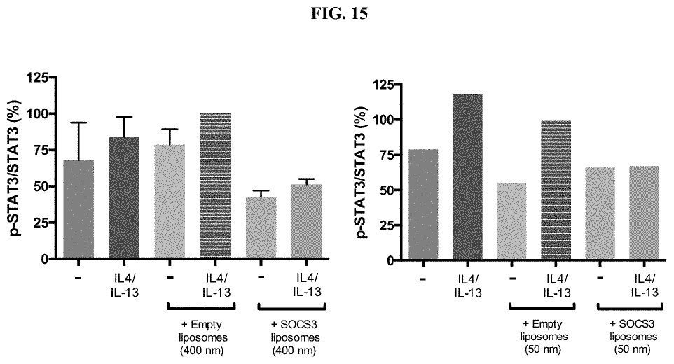

A similar, albeit more modest degree of inhibition, was observed by SOCS3-containing liposomes in IL-6-stimulated A549 human adenocarcinoma cells; 400 nm liposomes gave a ˜40% inhibition, which was comparable to that seen with conditioned medium from normal rat alveolar macrophages, whereas 50 nm liposomes gave a ˜15% inhibition (FIG. 13). IL-4 and IL-13 are prototypic Th2 cytokines that are pivotal drivers of allergic (so-called “type 2”) inflammation. These typically signal via STAT6, but can also activate STAT3 and NF-κB. BEAS-2b normal human bronchial epithelial cells were pretreated for 2 h with empty or SOCS-3 containing liposomes prior to addition of IL-4/IL-13. 1 hour thereafter, cells were harvested for analysis of transcription factor activation by western blot. 400 nm SOCS3-containing liposomes attenuated activation of STAT6 (FIG. 14, left) and NF-κB (FIG. 14, right), as compared to empty liposomes. Both 400 nm (FIG. 15, left) and 50 nm (FIG. 15, right) SOCS3-containing liposomes blunted IL-4/IL-13 induced activation of STAT3.

The data in FIGS. 12-15 demonstrate the ability of SOCS3-containing liposomes to exert broad suppressive actions on signaling responses in a variety of respiratory epithelial cells originating from different portions of the tracheobronchial tree and representing both normal and malignant phenotypes.

### In Vitro Effect of SOCS3-Containing Liposomes on Functional Responses in Epithelial Cells

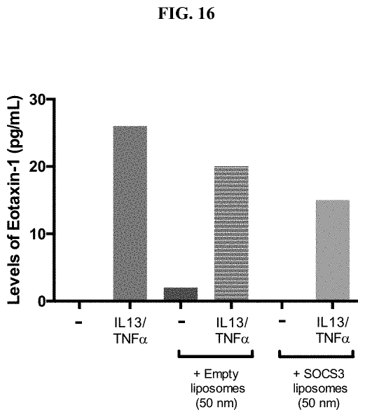

Experiments were conducted during development of embodiments herein to investigate the ability of liposomes to modulate BEAS-2b cell generation of the STATE-dependent chemokine eotaxin-1, which is important in eosinophil recruitment to the airways in allergic asthma. Experiments revealed that the combination of cytokines IL-13 and TNF-α was optimal for inducing eotaxin-1 protein (measured at 48 hours after cytokine addition by ELISA). As compared to empty liposomes, 50 nm SOCS3-containing liposomes blunted cytokine-induced eotaxin-1 generation (FIG. 16).

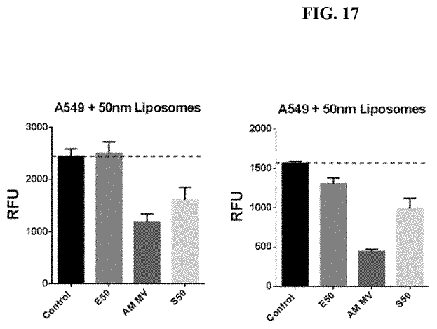

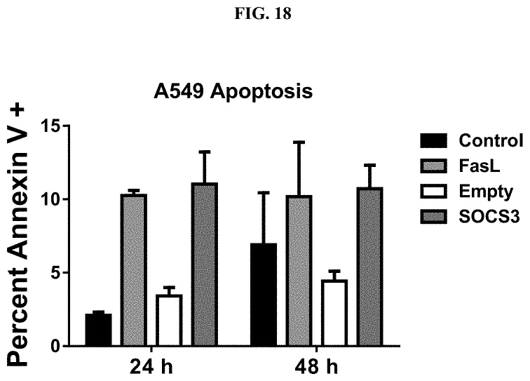

STAT3 is critical for tumor development and expansion. The effects of SOCS3-containing liposomes were assessed on two relevant processes contributing to tumor cell expansion, namely, proliferation and survival, in A549 human adenocarcinoma cells. FIG. 17 shows the effects of 50 nm SOCS3-containing liposomes on cell proliferation, measured by DNA binding at 72 hours after treatment, in 2 separate experiments (left and right). As compared to control (no liposomes), empty 50 nm liposomes had no effect on proliferation. By contrast, SOCS3 liposomes had a ˜40% inhibitory effect on cell proliferation in both experiments; this effect was somewhat more modest than that of the positive control, rat alveolar macrophage-derived microvesicles. Tumor cell expansion reflects not only proliferation, but also their ability to resist apoptosis. Experiments have also examined the effect of liposomes on A549 cell apoptosis. Cells were treated for either 24 hours or 48 hours with or without a known apoptosis-inducing agent, the ligand for the Fas death receptor, and apoptotic cells were quantified by their surface expression of phosphatidylserine, measured by annexin-V staining via flow cytometry (FIG. 18). Fas ligand robustly promoted apoptosis. Empty 50 nm liposomes had little effect. However, SOCS3-containing liposomes elicited apoptosis to the same degree as did Fas ligand.

The data in FIGS. 16-18 show that SOCS3-containing liposomes, once internalized by epithelial cells, modulate critical physiologic processes including cytokine generation, proliferation, and survival.

### In Vivo Effect of SOCS3-Containing Liposomes on Signaling and Functional Responses in Lung Tissue

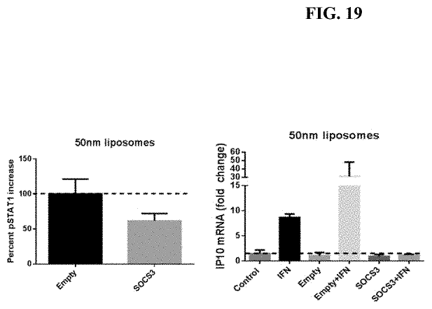

Delivery of alveolar macrophage-derived microvesicles to the mouse lung blunts cytokine-induced signaling in a SOCS3-dependent manner. Experiments were conducted during development of embodiments herein to investigate the in vivo impact of artificial liposome vesicles encapsulating SOCS3. The approximate amount of SOCS3 protein found in the macrophage-derived vesicles that were delivered to mice were used as a guide for dosing in the artificial vesicle studies. It was observed that the concentration of SOCS3 protein per vesicle is particularly important in these in vivo studies, since—for a given protein dose administered—the number of liposomes necessary to reach the target protein amount with highly-loaded vesicles will be insufficient in number to provide adequate distribution throughout the respiratory surface. Experiments were conducted during development of embodiments herein to assess the ability of liposomes to attenuate STAT1 phosphorylation and cytokine generation in response to administration of the pro-inflammatory cytokine, IFN-γ. Empty or SOCS3-containing 50 nm liposomes were administered oropharyngeally to C57BL/6 mice. 2 hours later, IFN-γ was administered by the same route. 1 hour thereafter, lungs were harvested and homogenates analyzed for STAT1 phosphorylation (by Western blot)(FIG. 19, left) and for mRNA levels of the STAT1-dependent chemokine gene, IP-10 (by RT-PCR)(FIG. 19, right). As compared to empty liposomes, SOCS3 liposomes attenuated STAT1 activation and markedly abrogated IP-10 induction.

These data demonstrate the capacity of SOCS3-containing liposomes to abrogate inflammatory responses in the lung in vivo.

### REFERENCES

The following references, some of which are cited above, are herein incorporated by reference in their entireties.
Akram, K. M., N. J. Lomas, N. R. Forsyth, and M. A. Spiteri. 2014. Alveolar epithelial cells in idiopathic pulmonary fibrosis display upregulation of TRAIL, DR4 and DR5 expression with simultaneous preferential over-expression of pro-apoptotic marker p53. Int J Clin Exp Pathol 7:552-564.
Alexander, W. S., and D. J. Hilton. 2004. The role of suppressors of cytokine signaling (SOCS) proteins in regulation of the immune response. Annu. Rev. Immunol. 22:503-529.
Aronoff, D. M., C. Canetti, and M. Peters-Golden. 2004. Prostaglandin E2 inhibits alveolar macrophage phagocytosis through an E-prostanoid 2 receptor-mediated increase in intracellular cyclic AMP. Journal of immunology 173:559-565.
Balter, M. S., G. B. Toews, and M. Peters-Golden. 1989. Multiple defects in arachidonate metabolism in alveolar macrophages from young asymptomatic smokers. The Journal of laboratory and clinical medicine 114:662-673.
Bendtsen, J. D., L. J. Jensen, N. Blom, G. Von Heijne, and S. Brunak. 2004a. Feature-based prediction of non-classical and leaderless protein secretion. Protein Eng. Des. Sel. 17:349-356.
Bendtsen, J. D., H. Nielsen, G. von Heijne, and S. Brunak. 2004b. Improved prediction of signal peptides: SignalP 3.0. J. Mol. Biol. 340:783-795.
Bernard, M. A., H. Zhao, S. C. Yue, A. Anandaiah, H. Koziel, and S. D. Tachado. 2014. Novel HIV-1 miRNAs stimulate TNFalpha release in human macrophages via TLR8 signaling pathway. PloS one 9:e106006.
Bourdonnay, E., C. H. Serezani, D. M. Aronoff, and M. Peters-Golden. 2012. Regulation of alveolar macrophage p40phox: hierarchy of activating kinases and their inhibition by PGE2. J. Leukoc. Biol. 92:219-231.
Brandes, M. E., and J. N. Finkelstein. 1989. Stimulated rabbit alveolar macrophages secrete a growth factor for type II pneumocytes. American journal of respiratory cell and molecular biology 1:101-109.
Brock, T. G., E. Maydanski, R. W. McNish, and M. Peters-Golden. 2001. Co-localization of leukotriene a4 hydrolase with 5-lipoxygenase in nuclei of alveolar macrophages and rat basophilic leukemia cells but not neutrophils. J. Biol. Chem. 276:35071-35077.
Brogan, P. A., V. Shah, C. Brachet, A. Harnden, D. Mant, N. Klein, and M. J. Dillon. 2004. Endothelial and platelet microparticles in vasculitis of the young. Arthritis Rheum. 50:927-936.
Canetti, C., D. M. Aronoff, M. Choe, N. Flamand, S. Wettlaufer, G. B. Toews, G. H. Chen, and M. Peters-Golden. 2006. Differential regulation by leukotrienes and calcium of Fc gamma receptor-induced phagocytosis and Syk activation in dendritic cells versus macrophages. J. Leukoc. Biol. 79:1234-1241.
Chauncey, J. B., M. Peters-Golden, and R. H. Simon. 1988. Arachidonic acid metabolism by rat alveolar epithelial cells. Lab. Invest. 58:133-140.
Choi, D. S., D. K. Kim, Y. K. Kim, and Y. S. Gho. 2013. Proteomics, transcriptomics and lipidomics of exosomes and ectosomes. Proteomics 13:1554-1571.
Chuquimia, O. D., D. H. Petursdottir, M. J. Rahman, K. Hartl, M. Singh, and C. Fernandez. 2012. The role of alveolar epithelial cells in initiating and shaping pulmonary immune responses: communication between innate and adaptive immune systems. PloS one 7:e32125.

Cosio, M. G., M. Saetta, and A. Agusti. 2009. Immunologic aspects of chronic obstructive pulmonary disease. The New England journal of medicine 360:2445-2454.
Dhillon, N. K., W. J. Murphy, M. B. Filla, A. J. Crespo, H. A. Latham, and A. O'Brien-Ladner. 2009. Down modulation of IFN-gamma signaling in alveolar macrophages isolated from smokers. Toxicol. Appl. Pharmacol. 237:22-28.
Duval, D., B. Reinhardt, C. Kedinger, and H. Boeuf. 2000. Role of suppressors of cytokine signaling (Socs) in leukemia inhibitory factor (LIF)-dependent embryonic stem cell survival. FASEB J. 14:1577-1584.
Gasser, O., C. Hess, S. Miot, C. Deon, J. C. Sanchez, and J. A. Schifferli. 2003. Characterisation and properties of ectosomes released by human polymorphonuclear neutrophils. Experimental cell research 285:243-257.
Greenhalgh, C. J., and W. S. Alexander. 2004. Suppressors of cytokine signalling and regulation of growth hormone action. Growth Horm. IGF Res. 14:200-206.
Guth, A. M., W. J. Janssen, C. M. Bosio, E. C. Crouch, P. M. Henson, and S. W. Dow. 2009. Lung environment determines unique phenotype of alveolar macrophages. American journal of physiology. Lung cellular and molecular physiology 296:L936-946.
Hasday, J. D., R. Bascom, J. J. Costa, T. Fitzgerald, and W. Dubin. 1999. Bacterial endotoxin is an active component of cigarette smoke. Chest. 115:829-835.
Holt, P. G. 1987. Immune and inflammatory function in cigarette smokers. Thorax 42:241-249.
Hugel, B., M. C. Martinez, C. Kunzelmann, and J. M. Freyssinet. 2005. Membrane microparticles: two sides of the coin. Physiology (Bethesda) 20:22-27.
Hussell, T., and T. J. Bell. 2014. Alveolar macrophages: plasticity in a tissue-specific context. Nature reviews. Immunology 14:81-93.
Hyde, D. M., N. K. Tyler, L. F. Putney, P. Singh, and H. J. Gundersen. 2004. Total number and mean size of alveoli in mammalian lung estimated using fractionator sampling and unbiased estimates of the Euler characteristic of alveolar openings. The anatomical record. Part A, Discoveries in molecular, cellular, and evolutionary biology 277:216-226.
Jo, D., D. Liu, S. Yao, R. D. Collins, and J. Hawiger. 2005. Intracellular protein therapy with SOCS3 inhibits inflammation and apoptosis. Nat. Med. 11:892-898.
John, G., K. Kohse, J. Orasche, A. Reda, J. Schnelle-Kreis, R. Zimmermann, O. Schmid, O. Eickelberg, and A. O. Yildirim. 2014. The composition of cigarette smoke determines inflammatory cell recruitment to the lung in COPD mouse models. Clinical science 126:207-221.
Jose, P., M. G. Avdiushko, S. Akira, A. M. Kaplan, and D. A. Cohen. 2009. Inhibition of interleukin-10 signaling in lung dendritic cells by toll-like receptor 4 ligands. Exp. Lung Res. 35:1-28.
Kelley, J. L., M. M. Rozek, C. A. Suenram, and C. J. Schwartz. 1987. Activation of human blood monocytes by adherence to tissue culture plastic surfaces. Exp. Mol. Pathol. 46:266-278.
MacKenzie, A., H. L. Wilson, E. Kiss-Toth, S. K. Dower, R. A. North, and A. Surprenant. 2001. Rapid secretion of interleukin-lbeta by microvesicle shedding. Immunity 15:825-835.
Mallat, Z., H. Benamer, B. Hugel, J. Benessiano, P. G. Steg, J. M. Freyssinet, and A. Tedgui. 2000. Elevated levels of shed membrane microparticles with procoagulant potential in the peripheral circulating blood of patients with acute coronary syndromes. Circulation 101:841-843.
Mause, S. F., and C. Weber. 2010. Microparticles: protagonists of a novel communication network for intercellular information exchange. Circ. Res. 107:1047-1057.
Nicholson, S. E., T. A. Willson, A. Farley, R. Starr, J. G. Zhang, M. Baca, W. S. Alexander, D. Metcalf, D. J. Hilton, and N. A. Nicola. 1999. Mutational analyses of the SOCS proteins suggest a dual domain requirement but distinct mechanisms for inhibition of LIF and IL-6 signal transduction. EMBO J. 18:375-385.
Nickel, W., and C. Rabouille. 2009. Mechanisms of regulated unconventional protein secretion. Nat. Rev. Mol. Cell Biol. 10:148-155.
Phipps, J. C., D. M. Aronoff, J. L. Curtis, D. Goel, E. O'Brien, and P. Mancuso. 2010. Cigarette smoke exposure impairs pulmonary bacterial clearance and alveolar macrophage complement-mediated phagocytosis of Streptococcus pneumoniae. Infection and immunity 78:1214-1220.
Qing, Y., and G. R. Stark. 2004. Alternative activation of STAT1 and STAT3 in response to interferon-gamma. J. Biol. Chem. 279:41679-41685.
Raposo, G., and W. Stoorvogel. 2013. Extracellular vesicles: exosomes, microvesicles, and friends. J Cell Biol 200:373-383.
Rubartelli, A., F. Cozzolino, M. Talio, and R. Sitia. 1990. A novel secretory pathway for interleukin-1 beta, a protein lacking a signal sequence. EMBO J. 9:1503-1510.
Sabat, R., G. Grutz, K. Warszawska, S. Kirsch, E. Witte, K. Wolk, and J. Geginat. 2010. Biology of interleukin-10. Cytokine Growth Factor Rev. 21:331-344.
Sadallah, S., C. Eken, and J. A. Schifferli. 2008. Erythrocyte-derived ectosomes have immunosuppressive properties. J. Leukoc. Biol. 84:1316-1325.
Shen, B., N. Wu, J. M. Yang, and S. J. Gould. 2011. Protein targeting to exosomes/microvesicles by plasma membrane anchors. J. Biol. Chem. 286:14383-14395.
Standiford, T. J., S. L. Kunkel, M. A. Basha, S. W. Chensue, J. P. Lynch, 3rd, G. B. Toews, J. Westwick, and R. M. Strieter. 1990. Interleukin-8 gene expression by a pulmonary epithelial cell line. A model for cytokine networks in the lung. The Journal of clinical investigation 86:1945-1953.
Starr, R., T. A. Willson, E. M. Viney, L. J. Murray, J. R. Rayner, B. J. Jenkins, T. J. Gonda, W. S. Alexander, D. Metcalf, N. A. Nicola, and D. J. Hilton. 1997. A family of cytokine-inducible inhibitors of signalling. Nature 387:917-921.
Takanashi, S., Y. Hasegawa, Y. Kanehira, K. Yamamoto, K. Fujimoto, K. Satoh, and K. Okamura. 1999. Interleukin-10 level in sputum is reduced in bronchial asthma, COPD and in smokers. The European respiratory journal 14:309-314.
Tamiya, T., I. Kashiwagi, R. Takahashi, H. Yasukawa, and A. Yoshimura. 2011. Suppressors of cytokine signaling (SOCS) proteins and JAK/STAT pathways: regulation of T-cell inflammation by SOCS1 and SOCS3. Arteriosclerosis, thrombosis, and vascular biology 31:980-985.
Thepen, T., G. Kraal, and P. G. Holt. 1994. The role of alveolar macrophages in regulation of lung inflammation. Annals of the New York Academy of Sciences 725:200-206.
Thery, C., S. Amigorena, G. Raposo, and A. Clayton. 2006. Isolation and characterization of exosomes from cell culture supernatants and biological fluids. Curr. Protoc. Cell Biol. Chapter 3:Unit 3 22.
Wang, J. W., C. M. Gijsberts, A. Seneviratna, V. C. de Hoog, J. E. Vrijenhoek, A. H. Schoneveld, M. Y. Chan, C. S. Lam, A. M. Richards, C. N. Lee, A. Mosterd, S. K. Sze, L. Timmers, S. K. Lim, G. Pasterkamp, and D. P. de Kleijn. 2013. Plasma extracellular vesicle protein content for diagnosis and prognosis of global cardiovascular disease. Netherlands heart journal: monthly journal of the Netherlands Society of Cardiology and the Netherlands Heart Foundation 21:467-471.
Westphalen, K., G. A. Gusarova, M. N. Islam, M. Subramanian, T. S. Cohen, A. S. Prince, and J. Bhattacharya. 2014. Sessile alveolar macrophages communicate with alveolar epithelium to modulate immunity. Nature 506:503-506.
White, G. E., A. Cotterill, M. R. Addley, E. J. Soilleux, and D. R. Greaves. 2011. Suppressor of cytokine signalling protein SOCS3 expression is increased at sites of acute and chronic inflammation. Journal of molecular histology 42:137-151.
Yoshimura, A., T. Naka, and M. Kubo. 2007. SOCS proteins, cytokine signalling and immune regulation. Nature reviews. Immunology 7:454-465.
Yoshimura, A., T. Ohkubo, T. Kiguchi, N. A. Jenkins, D. J. Gilbert, N. G. Copeland, T. Hara, and A. Miyajima. 1995. A novel cytokine-inducible gene CIS encodes an SH2-containing protein that binds to tyrosine-phosphorylated interleukin 3 and erythropoietin receptors. EMBO J. 14:2816-2826.
Yu, H., H. Lee, A. Herrmann, R. Buettner, and R. Jove. 2014. Revisiting STAT3 signalling in cancer: new and unexpected biological functions. Nature Reviews. Cancer 14:736-746.
Zeitvogel, J., A. Dalpke, B. Eiz-Vesper, M. Kracht, O. Dittrich-Breiholz, T. Werfel, and M. Wittmann. 2012. Human primary keratinocytes show restricted ability to up-regulate suppressor of cytokine signaling (SOCS)3 protein compared with autologous macrophages. J. Biol. Chem. 287:9923-9930.

1. A composition comprising synthetic vesicles encapsulating one or more SOCS polypeptides.

2. The composition of claim 1, wherein the synthetic vesicles comprise one or more lipids selected from the group consisting of egg phosphatidylcholine (EPC), egg phosphatidylglycerol (EPG), egg phosphatidylinositol (EPI), egg phosphatidylserine (EPS), phosphatidylethanolamine (EPE), phosphatidic acid (EPA), soy phosphatidylcholine (SPC), soy phosphatidylglycerol (SPG), soy phosphatidylserine (SPS), soy phosphatidylinositol (SPI), soy phosphatidylethanolamine (SPE), soy phosphatidic acid (SPA), hydrogenated egg phosphatidylcholine (HEPC), hydrogenated egg phosphatidylglycerol (HEPG), hydrogenated egg phosphatidylinositol (HEPI), hydrogenated egg phosphatidylserine (HEPS), hydrogenated phosphatidylethanolamine (HEPE), hydrogenated phosphatidic acid (HEPA), hydrogenated soy phosphatidylcholine (HSPC), hydrogenated soy phosphatidylglycerol (HSPG), hydrogenated soy phosphatidylserine (HSPS), hydrogenated soy phosphatidylinositol (HSPI), hydrogenated soy phosphatidylethanolamine (HSPE), hydrogenated soy phosphatidic acid (HSPA), dipalmitoylphosphatidylcholine (DPPC), dimyristoylphosphatidylcholine (DMPC), dimyristoylphosphatidylglycerol (DMPG), dipalmitoylphosphatidylglycerol (DPPG), distearoylphosphatidylcholine (DSPC), distearoylphosphatidylglycerol (DSPG), dioleylphosphatidyl-ethanolamine (DOPE), palmitoylstearoylphosphatidyl-choline (PSPC), palmitoylstearolphosphatidylglycerol (PSPG), mono-oleoyl-phosphatidylethanolamine (MOPE), cholesterol, ergosterol, lanosterol, tocopherol, ammonium salts of fatty acids, ammonium salts of phospholipids, ammonium salts of glycerides, myristylamine, palmitylamine, laurylamine, stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethyl ammonium chloride (DOTMA), 1,2-bis(oleoyloxy)-3-(trimethylammonio)propane (DOTAP), phosphatidyl-glycerols (PGs), phosphatidic acids (PAs), phosphatidylinositols (PIs), phosphatidyl serines (PSs), distearoylphosphatidylglycerol (DSPG), dimyristoylphosphatidylacid (DMPA), dipalmitoylphosphatidylacid (DPPA), distearoylphosphatidylacid (DSPA), dimyristoylphosphatidylinositol (DMPI), dipalmitoylphosphatidylinositol (DPPI), distearoylphospatidylinositol (DSPI), dimyristoylphosphatidylserine (DMPS), dipalmitoylphosphatidylserine (DPPS), distearoylphosphatidylserine (DSPS), and mixtures thereof.

3. The composition of claim 1, wherein one or more SOCS polypeptides comprises a SOCS1 polypeptide.

4. The composition of claim 3, wherein the SOCS1 polypeptide comprises greater than 60% sequence identity to SEQ ID NO: 1.

5. The composition of claim 1, wherein one or more SOCS polypeptides comprises a SOCS3 polypeptide.

6. The composition of claim 3, wherein the SOCS3 polypeptide comprises greater than 60% sequence identity to SEQ ID NO: 3.

7. The composition of claim 1, wherein one or more SOCS polypeptides attenuate STAT phosphorylation.

8. The composition of claim 1, wherein the synthetic vesicles are formulated for pulmonary administration.

9. The composition of claim 8, wherein the synthetic vesicles are formulated for inhalation by a subject.

10. The composition of claim 8, wherein the synthetic vesicles are aerosolized.

11. A method of treating a pulmonary condition or disease comprising administering a composition of claim 1 to a subject suffering from the pulmonary condition or disease.

12. The method of claim 11, wherein the pulmonary condition or disease is characterized by inflammation.

13. The method of claim 12, wherein administration of a composition of claim 1 results in decreased inflammation.

14. A method of treating lung cancer comprising administering a composition of claim 1 to a subject suffering from lung cancer.
